SEQUENCE LISTING (8 sequences)

SEQ ID NO: 1 - protein, 42 aa, Homo sapiens [sequence omitted]

SEQ ID NO: 2 - protein, 16 aa, Homo sapiens
QSHYRHISPAQVHHQK

SEQ ID NO: 3 - protein, 16 aa, Artificial Sequence
  D-enantiomeric peptide D3
RPRTRLHTHRNRHHQK

SEQ ID NO: 4 - protein, 7 aa, Artificial Sequence
  peptide residue
CKFFVLK

SEQ ID NO: 5 - protein, 7 aa, Artificial Sequence
  peptide
KKFFVLK

SEQ ID NO: 6 - protein, 9 aa, Artificial Sequence
  peptide
KKFFVLKGK

SEQ ID NO: 7 - protein, 9 aa, Artificial Sequence
  peptide
KKFFVLKGC

SEQ ID NO: 8 - protein, 8 aa, Artificial Sequence
  peptide
KKFFVLKG

(19) United States
(12) Patent Application Publication
(10) Pub. No.: US 2019/0094246 A1
(43) Pub. Date: Mar. 28, 2019

(54) DETECTION OF NEURODEGENERATIVE DISEASES

(71) Applicants: AMONETA DIAGNOSTICS SAS, Huningue (FR); JPT PEPTIDE TECHNOLOGIES GMBH, Berlin (DE); CNRS - Centre National de la Recherche Scientifiqu, Paris Cedex 16 (FR); Université de Strasbourg, Strasbourg Cedex (FR)

(72) Inventors: Saliha MOUSSAOUI, Bartenheim (FR); Dirk WILDEMANN, Halle (Saale) (DE); Holger WENSCHUH, Berlin (DE); Karsten SCHNATBAUM, Berlin (DE); Gilles ULRICH, Souffelweyersheim (FR); Jean DE BARRY, Strasbourg (FR); Corinne MBEBI-LIEGEOIS, Strasbourg (FR); Hueseyin FIRAT, Huningue (FR)

(21) Appl. No.: 16/083,440

(22) PCT Filed: Mar. 7, 2017
(86) PCT No.: PCT/IB2017/051343

§ 371 Date: Sep. 7, 2018

Related U.S. Application Data
(60) Provisional application No. 62/304,864, filed on Mar. 7, 2016.

(51) Int. Cl. G01N 33/68; C07K 14/47; C07F 5/02

(52) U.S. Cl. CPC G01N 33/6896; C07K 14/4711; C07F 5/02; G01N 2800/50; G01N 2333/912; G01N 2800/2821; G01N 2333/4709

(57) ABSTRACT

The present invention relates to a biomarker, and/or methods including a non-invasive in vitro method using this biomarker, for diagnosing or monitoring the development or the progression of Alzheimer's disease (AD) or a disease or disorder associated with β-amyloid peptide (Aβ) deposition or tau hyperphosphorylation or a disease or disorder characterized by a proteinopathy implicating abnormalities in protein kinase C (PKC).

### CROSS REFERENCE TO RELATED APPLICATIONS

This application claims the benefit and priority of U.S. provisional application 62/304,864, filed on Mar. 7, 2016.

### FIELD OF THE INVENTION

The present invention relates to a biomarker, and/or methods including a non-invasive in vitro method using this biomarker, for diagnosing or monitoring the development or the progression of Alzheimer's disease (AD) or a disease or disorder associated with β-amyloid peptide (Aβ) deposition or tau hyperphosphorylation or a disease or disorder characterized by a proteinopathy implicating abnormalities in protein kinase C (PKC).

### BACKGROUND

AD is a chronic neurodegenerative disease characterized by progressive loss of cognitive function and pathologically by extracellular deposition of amyloid-beta peptides (Aβ42 and its shorter fragments, e.g. Aβ40 and Aβ38) and intracellular deposition of hyper-phosphorylated tau protein in neurofibrillary tangles in the brain and progressive neuronal degeneration (Marcus et al. J Neurogenet. 2011; 25(4):127-33; Gotz et al, Br J Pharmacol. 2012; 165(5):1246-59).

Currently, there is no cure for AD. Symptomatic treatments exist for this disease all trying to counterbalance neurotransmitter's disturbance. Although a number of new potential disease-modifying therapeutic candidates are currently being studied in clinical trials, none has been approved yet. From 2002 to 2012, there was a failure of 99.6% of AD clinical trials that were 289 phase 2 and 3 trials on symptomatic agents (36.6%), disease-modifying small molecules (35.1%) and disease-modifying immunotherapies (18%) (Cummings et al., Expert Rev ClinPharmacol. 2014; 7(2):161-5). Among the strategies proposed to improve the success rate for AD drug development, are: a) intervening earlier in the disease process before neurodegeneration begins, b) identifying and developing relevant biomarkers for early diagnosis, suitable for stratification of subject populations and for longitudinal monitoring of drug efficacy in clinical trials.

Current diagnosis of the disease remains uncertain (Dubois et al Lancet Neurol. 2014; 13(6):614-29) and it is based on combination of a battery of clinical and neuropsychological tests and neuroimaging, mainly using structural Magnetic Resonance Imaging (MRI) and/or glucose metabolism using Positron emission tomography (PET) of fluorodeoxyglucose F18 (¹⁸F-FDG), with an accuracy of less than 70%-85% depending on the method and the severity stage of the disease (Frisoni et al., Nat Rev Neurol. 2010; 6(2): 67-77; Tahmasian et al., J Nucl Med. 2016; 57(3):410-5).

Sometimes, detection using immunoassay methods of Aβ peptides (Aβ42 or Aβ40) and tau or phosphorylated-tau in the cerebrospinal fluid (CSF) is additionally performed (Sunderland et al., JAMA. 2003; 289(16):2094-2103), but these CSF tests, although relevant for AD pathology and detection of early AD, request a lumbar puncture which is an invasive procedure, thus dramatically limiting their use. Longitudinal confirmatory diagnosis test is essential for applications to early detection of the preclinical stages of AD and mild cognitive impairment (MCI; defined as an early stage during which the first subtle symptoms manifest) and thus detecting early and calculating the risk of conversion of MCI into AD. Due to its limits (invasive), a CSF test is not suitable for use as a biomarker for early diagnosis in preclinical AD stages before the symptoms appear, nor it is suitable as a companion biomarker test that needs to be repeatedly practiced in same subjects recruited in longitudinal clinical trials. The methods of the present invention, in particular the in vitro method does not suffer from these limitations of CSF biomarkers. The in vitro method of the invention can be used without an invasive procedure for sampling as it can be practiced on patient blood cells, and it can be practiced at different time-points, thus suitable to monitor the disease progression from preclinical to clinical AD, as well as for repeated monitoring of drug responses in longitudinal trials.
Detection using antibodies and immunoassay methods of the biomarkers (Aβ42, Aβ40, tau or phosphorylated-tau) in serum or plasma or blood cell membranes as a non-invasive procedure lacked till now the specificity and sensitivity necessary to identify AD subjects and the corresponding methods were unsuccessful in producing a reliable and accurate assay for diagnosis of AD (Blennow K et al, Cold Spring Harb Perspect Med. 2012 September; 2(9): a006221), including when using blood cell membranes (Pesini P et al., 2009, 2d Conf. Clin Mats on Alzheimer's, Las Vegas, USA, November 2009; Frankfort et al., 2008, Curr Clin Pharmacol 3(2): 123-31). Indeed, the primary use of an antibody directed against Aβ peptide as performed by Pesini et al (2009) does not allow detection of the biomarker of the present invention.
Recently, in vivo molecular neuroimaging methods relevant for AD pathology are being developed, notably with amyloid-beta (Aβ) ligands for Positron Emission Tomography (PET) including ¹⁸F-florbetapir and ¹⁸F-flutemetamol (Choi et al., 2009, J Nucl Med. 2009; 50(11):1887-94; Wong et al., 2010, J Nucl Med. 51(6):913-20). However, their diagnosis accuracy is still being studied for further understanding. Indeed, PET imaging using these Aβ ligands showed that up to 30% of subjects diagnosed with AD show a negative Aβ scan and that up to 35% of subjects with normal cognition status show a positive Aβ scan (Chételata G et al, NeuroImage Clinical, 2 (2013):356-65. Thus, an Aβ PET such as that using ¹⁸F-florbetapir and ¹⁸F-flutemetamol is sensitive to detect early brain amyloidosis but they lack specificity to differentially detect specifically patients with AD nor to predict in subjects with mild cognitive impairment the conversion of MCI to AD. PET Aβ scan was recently used to guide for recruitment of subjects in the desired cohorts of patients with early AD in some clinical trials; However, the therapeutic candidate anti-Aβ antibody Solanezumab tested in patients with mixed mild-to-moderate AD failed to show clear efficacy on cognitive decline.

Overall, the existing tests either lack an easy accessibility and simplicity for use for diagnosis of the large AD population and/or lack accuracy (sensitivity and specificity). This represents a major impediment and bottleneck to develop reliable and rapid diagnosis test for AD. Another impediment is the identification of a biomarker that does not require invasive sample collecting, such as a spinal tap. The lack of such an accessible, sensitive and specific biomarker impedes the development of therapies and drugs for AD or for the studies on pathological processes triggering AD or involved in the progression of AD. Today, clearly there is an unmet need for an accurate and relevant biomarker test for differential diagnosis of AD including preclinical and early AD and for applications in drug development (patient stratification and monitoring drug efficacy in clinical trials).

Furthermore, results described in a number of publications converge to suggest that among kinases implicated in the phosphorylation status of tau and the beta-amyloid precursor protein (APP), the precursor of Aβ peptides; the protein Kinase C (PKC) may play a key role (Jean de Barry et al, 2010, Exp. Gerontol, 45: 64-69):
PKC is a group of 12 PKC kinase isoforms, expressed in the body cells. PKC activation implies its phosphorylation and its conformational change and translocation of the inactive cytosolic enzyme towards the cellular membrane where it interacts with diacylglycerol DAG) and its exogenous activators such as phorbol ester. Phorbol ester-induced alpha-secretase activation involves translocation of PKC from the cytosol to the membrane compartment.
The activation of PKC fosters the non-amyloidogenic α-secretase (ADAM10) cleavage pathway of APP, thus enhances Aβ degradation, thereby reducing Aβ production and potentially amyloid plaques. The capability of PKC of regulating ADAM10 activity may be related to direct or indirect modification of ADAM10 function or subcellular localization (C. Saraceno et al, 2014, Cell Death Dis. 5(11): e1547).
In AD, PKC alterations described are decreased levels, activity and translocation and involvement in alteration of the transduction system, which all may favor reduced Aβ degradation, enhanced Aβ production and amyloid plaque formation.
On the other hand, Aβ can inactivate PKC: Aβ contains a putative PKC pseudo-substrate site (A1328-30) critical for A13-PKC interaction. A13 can degrade PKC in human fibroblasts. Aβ reduces activated forms of PKC and inhibits its phorbol ester-induced membrane translocation. Aβ also enhances cellular Ca2+ which may inactivate PKC. Furthermore, Aβ induces a number of cellular events including activation of kinases that phosphorylates tau such as ERK1/2 and GSK-3β. PKC can inhibit GSK-3β directly, thus reducing tau phosphorylation.
Thus, a deficiency in PKC as seen in AD may play a role in stimulating both types of lesions by enhancing:
   (i) the production of endogenous Aβ→amyloid plaques
   (ii) tau phosphorylation→neurofibrillary tangles

Besides for AD which is characterized by both Aβ deposits, tau deposits and PKC abnormalities, for other neurodegenerative diseases, there is also unmet need for non-invasive peripheral biomarkers for diagnosis. These diseases include:
diseases characterized by Aβ deposits, without or less neurofibrillary tangles, e.g. vascular dementia (VaD),
diseases characterized by tau hyperphosphorylation, without or less Aβ deposits, e.g. frontotemporal dementia (FTD), progressive supranuclear palsy (PSP) or corticobasal degeneration (CBD),
otherproteinopathies, associated with abnormal changes in PKC proteins or enzymatic activities.

There is a high need for a sensitive and specific biomarker suitable for methods including in vitro method that does not require invasive procedure as lumbar puncture for sample collecting, but non-invasive collection of samples, and which can be based on blood and other peripheral cells of the body. Such a biomarker is not only required to identify early AD but also to contribute to develop therapies and drugs for AD or for the pathological processes triggering AD or involved in the progression of AD.

### SUMMARY OF THE INVENTION

The non-invasive tests performed according to the invention show that in blood cells, e.g. erythrocytes, when incubated with a selected compound of the invention (e.g., Compound of Formula I), this compound is capable of subsequently interacting and/or binding to the Aβ biomarker endogenously present in the cells and subsequently make the cells stained with measurable fluorescence intensity using for example the flow cytometry technique or a microscopy device equipped with a spectral detector. This makes it possible to quantify the human Aβ peptides endogenously present in the circulating blood cells such as erythrocytes of a subject. Likewise, when incubated with another selected compound of the invention, this specific compound is capable of subsequently interacting with its biomarker PKC endogenously present in the membrane and cytosol of cells and subsequently makes the cells stained with measurable fluorescence intensity. This makes it possible to quantify the human PKC endogenously present in the circulating cells of a subject.

More specifically, the method of diagnosis of a neurodegenerative disease such as AD implements the original detection method according to the invention; it makes it possible to evaluate the biomarkers Aβ peptide and PKC in a biological sample where the biological sample contains the cells, for example the blood for the erythrocytes, and the compound.

The present invention relates to novel compounds enabling to measure the respective endogenous biomarkers (AO) peptide and/or the protein kinase C (PKC) relevant for the pathology of a neurodegenerative disease, in particular Alzheimer's disease (AD). The present invention relates further to their use as biomarkers, their combination and corresponding assays using one or more of these new biomarkers. These compounds may be used to measure, detect, and/or diagnose neurodegenerative diseases (e.g., Alzheimer's Disease).

These endogenous biomarkers are also detectable and measurable using the compounds of the present invention either a) in vitro in cultured cellular models, and in peripheral circulating cells of animal models of a neurodegenerative disease in particular AD, orb) ex-vivo or in vivo in the brain of animal models neurodegenerative disease such as AD.

Since the compounds of the present invention are specific to the endogenous biomarkers Aβ peptides and PKC, that are relevant for the physiopathology of the disease and reports on disease progression, the present invention also relates to their applications in early diagnosis of neurodegenerative disease in particular AD and differential diagnosis purposes, in treatment monitoring, in preclinical and clinical drug development and in developing new therapeutic pathways.

In one aspect the invention is directed to a method of detecting an endogenous biomarker of a neurodegenerative disease (e.g., Alzheimer's disease), wherein the method comprises: a.) obtaining a sample (e.g., blood sample) from a subject; b.) providing the sample one of the novel compounds of any of a compound of Formula I (e.g., any of Formula Ia, Ib, Ic), wherein the compound associates with (Aβ) peptide and/or the protein kinase C (PKC); and c.) detecting the association of the compound of any of a compound of Formula I, and the endogenous biomarker (e.g., (Aβ) peptide and/or the protein kinase C (PKC)).

In one aspect the invention is directed to a method of diagnosing a neurodegenerative disease (e.g., Alzheimer's Disease) in a subject, wherein the method comprises an endogenous biomarker (e.g., (Aβ) peptide and/or the protein kinase C (PKC)) of a neurodegenerative disease (e.g., Alzheimer's disease), wherein the method comprises: a.) obtaining a sample (e.g., blood sample) from a subject; providing the sample with one of the novel compounds of any of a compound of Formula I (e.g., any of Formula Ia, Ib, Ic), wherein the compound associates with the endogenous biomarker; c.) detecting the association of the compound of any of a compound of Formula I, and the endogenous biomarker (e.g., (Aβ) peptide and/or the protein kinase C (PKC)); d.) comparing the sample to a reference standard and e.) determining whether the subject has a neurodegenerative disease based upon the level and/or presence of the biomarker.

### DESCRIPTION OF THE FIGURES

FIG. 1: Represents the synthesis of Alanine-loaded TentaGel R PHB resin performed using a standard procedure for resin loading as indicated by the procedure. Key: DIC:DiisopropylcarbodiimideNMI: N-Methyl imidazole

FIG. 2: Represents a procedure for peptide synthesis described in Ex. 1 performed using an ABI synthesizer of Applied Biosystems®. Key: Fmoc-Xaa-OH:Fmoc-protectedaminoacid building block, HATU: 1-[Bis(dimethylamino)methylene]-1H-1,2,3-triazolo[4,5-b]pyridinium3-oxid hexafluorophosphate, NMM:N-Methylmorpholine, TFA:Trifluoroaceticacid, EDT: 1,2-Ethanedithiol, TIPS:Triisopropylsilane, ACN:Acetonitrile

FIG. 3: Represents the peptide synthesis described in Ex. 2 performed using an ABI synthesizer of Applied Biosystems®.

FIG. 4: Demonstrates a dose-dependent staining of RBCs (index MEDFI) with compound of Ex.1 (FIG. 4A) or compound of Ex. 9 (FIG. 4B).

FIG. 5: is a staining of RBCs (index MEDFI) with compound of Ex. 1 (FIG. 5A) or compound of Ex.9 (FIG. 5B) (circles) and effect of these two compounds on RBC survival (squares).

FIG. 6: demonstrates the LOD and LLOQ for the staining of RBCs from human subjects with compound of Ex. 1 (FIG. 6A) and compound of Ex.9 (FIG. 6B).

FIG. 7: demonstrates the effect of concentrations of RBCs on the intensity of their staining by compound of Ex.1 (FIG. 7A) or compound of Ex.9 (FIG. 7B) used separately.

FIG. 8: demonstrates results of a study in transgenic APP/PS1 mice versus age-matched Wild-Type mice for 1 μM compound of Ex. 1 (FIG. 8A), and for 1 μM compound of Ex. 9 (FIG. 8B).

FIG. 9: demonstrates the correlation between the RBC staining with 1 μM compound of Ex. 3a and the RBC staining with 1 μM compound of Ex. 3b (left), between the RBC staining with 0.3 μM compound of Ex. 1 and 0.6 μM compound of Ex. 3f (middle) and between the RBC staining with 0.3 μM compound of Ex. 3b and 0.6 μM compound of Ex. 3f with a correlation factor of 0.809, 0.869 and 0.936, respectively.

FIG. 10: demonstrates the correlation between the cerebrospinal fluid level of A042 (ng/L) and the level of staining of RBCs (MEDFI) with compound of Ex.3a at different concentrations, in 8-14 Alzheimer patients. The correlation factor is 0.71 when the compound is tested at 1 μM.

FIG. 11: demonstrates the correlation between the cerebrospinal fluid level of A042 (ng/L) and the level of staining of RBCs (MF: mean fluorescence) with compound of Ex. 3f (FIG. 11A), compound of Ex. 3a (FIG. 11B) and compound of Ex. 3b (FIG. 11C) tested at a concentration of 1 μM in 8 to 14 Alzheimer patients.

FIG. 12: demonstrates: a) the RBC staining with 1 μM compound of Ex. 9 (raw data, top left graph) sorted out from low to highest level of mean fluorescence staining, b) the CSF levels of phosphorylated tau in the same patients (top right graph), c) the inverse correlation between the level of RBC staining with compound of Ex. 9 tested at 1 μM (Index MEDFI, relative mean normalization, X axis) and the level of CSF phosphorylated-tau (ng/L, Y axis) in 19 Alzheimer patients; the correlation coefficient being equal to −0.821 (graph at the bottom).

### DETAILED DESCRIPTION

### I. Compounds of the Invention

Accordingly, the present invention provides a compound of Formula I

P—Ar—R—X-T (I)

wherein
P is a residue of formula (a)

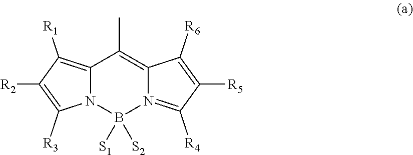

wherein
each of R₁, R₂, R₃, R₄, R₅ and R₆ is, independently, H or C₁-C₁₀alkyl, and
each of S₁ and S₂ is, independently, a hydrophilic group of formula —C≡C-L-A wherein L is a single bond, C₂-C₄alkenylene or linear, branched saturated C₂-C₂₀ carbon chain interrupted by 1 to 10 oxygen atoms; and A is C₁-C₄ alkyl, a phosphate group or a sulfonate group;
Ar is C₅-C₁₄arylene or heteroarylene on which —R—X-T is in ortho, meta or para position;
R is —CO—NH—, —NH—CO—, —CH₂—CO—NH— or —CH₂—NH—CO—; and
either X is a spacer serving to distance the residue T from the residue P without affecting the fluorescence of P and the biological activity of T, X being covalently bound through —S— to the a side chain of an amino-acid forming part of the residue T; and T is a peptidic residue selected from a residue of

An Aβ peptide,
(SEQ ID NO: 2)
QSHYRHISPAQVHHQK,
(SEQ ID NO: 3)
RPRTRLHTHRNRHHQK,
(SEQ ID NO: 4)
CKFFVLK-NH₂
(SEQ ID NO: 5)
KKFFVLK-NH₂,
(SEQ ID NO: 6)
KKFFVLKGK-NH₂,
and
(SEQ ID NO: 7)
KKFFVLKGC-NH₂

or a derivative or a structural analog thereof, comprising at least one amino acid with an a side chain;
   or X is a spacer serving to distance the residue T from the residue P without affecting the fluorescence of P and the biological activity of T; and
   T is a group of formula (b)

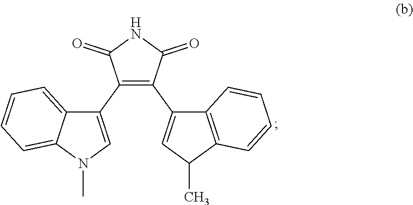 [SMILES: CC1C=C(C2=C(c3cn(C)c4ccccc34)C(=O)NC2=O)c2ccccc21]

In one aspect, P as described in Formula I has the properties of a fluorophore.

In one aspect, a suitable derivative or structural analog of a peptidic residue T, is e.g., the residue of a peptide wherein a Lys is substituted by Cys, or another amino-acid is substituted by Lys or Cys at any place of its chain, the resulting residue T retaining properties of binding to the endogenous biomarker Aβ.

In one aspect, when T is a peptidic residue as defined above, T is preferably the residue of a peptide comprising at least one Cys and/or Lys, the side chain thereof being bound to the spacer X through a —S— group. The a side chain may be e.g., a residue such as —(CH₂—)ₘ—S—; or —(CH₂—)_{m′}—NH—CO—(CH₂)ₙ—S— wherein m is 1, 2 or 3, m′ is 1, 2, 3, 4 or 5 and n is 1 or 2.

In one aspect, preferably the spacer X as described above is bound through —S— to the side chain of a Lys moiety, the side chain being preferably a residue —(CH₂—)₄—NH—CO—(CH₂)ₙ—S—.

In another aspect, when T is the residue of an Aβ peptide as defined above, it is preferably the residue of an Aβ peptide comprising at least 4, e.g. 6 contiguous amino acids of Aβ1-42 of a mammalian Aβ peptide, preferably human Aβ peptide comprising a Lys in position 16. The sequences of human Aβ1-42 peptide is shown in SEQ ID NO: 1. T can be a residue formed from specific Aβ peptides, e. g. human Aβ1-42, human Aβ1-40, human Aβ1-38, human Aβ1-16, human Aβ10-35, human Aβ14-25,human Aβ16-40, human Aβ16-42 or human Aβ25-35, or such an Aβ peptide wherein Lys in position 16 is replaced by Cys. peptide in different conformational states, in monomeric or in multimeric forms, or modified to change its conformational state or to form monomers or oligomers, for example by variation of salt, pH, temperature or surfactant concentration, may be employed. When T is a peptidic residue as indicated above, the spacer X is preferably linked to the side chain of a Lys moiety, whether comprised in the peptidic chain or being terminal.

The peptidic residues (SEQ ID NO: 4-7) CKFFVLK-NH₂, KKFFVLK-NH₂, KKFFVLKGK-NH₂, and KKFFVLKGC-NH₂ as T are based on the core sequence of Aβ16-20: KLVFF. These sequences of SEQ ID NO: 4-7 may be considered artificial or engineered.

In one aspect, when T is a residue of an Aβ peptide as defined above, it is preferably human Aβ1-40, human Aβ1-38 or human Aβ1-16 wherein Lys 16 is substituted as indicated above or wherein Lys16 is replaced by Cys 16.

In another aspect, T is preferably a residue of QSHYRHISPAQVHHQK or RPRTRLHTHRNRHHQK wherein Lys 16 is substituted as indicated above or wherein Lys 16 is replaced by Cys 16.

In one aspect, T is preferably a residue of CKFFVLK-NH₂, KKFFVLK-NH₂, KKFFVLKGK-NH₂, or KKFFVLKGC-NH₂,or a derivative or structural analog thereof, e.g. substituted in position 1 by C₈-C₁₆alkanoyl or alkenoyl, e.g. palmitoyl.

QSHYRHISPAQVHHQK is also known as D-enantiomeric peptide D1 and RPRTRLHTHRNRHHQK is also known as D-enantiomeric peptide D3. These peptides are discussed, for example, Bartnik et al., REJUVENATION RESEARCH Volume 13, Number 2-3, (2010), the contents of which are incorporated herein by reference.

In another aspect of Formula I, group of formula (b) as T has the properties of a PKC substrate.

C₁-C₁₀alkyl as R₁, R₂, R₃, R₄, R₅ or R₆ is preferably C₁-C₄alkyl. Linear or branched saturated C₂-C₂₀ carbon chain interrupted by 1 to 10 oxygen atoms as L may be a poly(ethylene oxide) or a poly(propylene oxide), the unit thereof repeating between 1 and 6 times. Preferably L is C₂-C₆alkylene optionally interrupted by 1 or 2 oxygen atoms.

C₅-C₁₄arylene as Ar is preferably phenylene. Example of heteroarylene as Ar is e.g. a divalent residue of pyridine. The moiety —R—X-T is preferably in para position.

In one aspect of Formula I as described herein, X as a spacer is preferably C₂-C₆alkylene or a residue of formula (c)

—R₇-R₈— (c)

wherein
R₇ is heteroC₄₋₇cycloalkylene, e.g. bearing a nitrogen atom as heteroatom, preferably a divalent group of formula

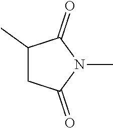

and
R₈ is a branched or linear C₁-C₆alkylene.
When T is a peptidic residue as indicated above and described throughout, X is preferably a residue of formula (c).
When T is a residue of formula (b), X is preferably C₂-C₆alkylene.
Preferably P is a symmetrical residue, particularly a residue wherein R₁ and R₆ are identical, R₂ and R₅ are identical, R₃ and R₄ are identical and S₁ and S₂ are identical. Preferably A is C₁-C₄ alkyl, particularly methyl.

In one aspect, a preferred group of compounds of Formula I are the compounds of formula Ia

P—Ar—R—Xₐ-Tₐ (Ia)

wherein P, Ar and R are as defined above,
Xₐ is a spacer serving to distance the residue Tₐ from the residue P without affecting the fluorescence of P and the biological activity of Tₐ, Xₐ being covalently bound through —S— to the a side chain of an amino-acid forming part of the residue Tₐ; and
Tₐ is a peptidic residue T as defined above (e.g., a peptidic residue or a group of formula (b); e.g., compounds of formula Ia are the compounds wherein T is a peptidic residue).

In one aspect of a compound of formula Ia, R is preferably —CO—NH—, —NH—CO— or —CH₂—NH—CO—.Xₐ has preferably one of the significances given above for X when T is a peptidic residue.

In another aspect of the invention, the compounds of formula Ia, are able to bind to a non-fluorinated dipyrromethene-boron on a peptide residue in an efficient way.

In one aspect, another group of preferred compounds of Formula I (as described herein) are the compounds of formula Ib

P—Ar—R_{b}—X_{b}-T_{b} (Ib)

wherein P and Ar are as defined above, R_{b} is —CO—NH— or —CH₂—CO—NH—; X_{b} is C₂-C₅ alkylene and T_{b} is a group of formula (b) as indicated above.

### Preferably R_{b} is —CO—NH—.

In one aspect the compound of Formula I is a compound of formula Ia, wherein formula Ia is compound of Example 1 described herein:

H-DAEFRHDSGYEVHHQ-X-LVFFAEDVGSNKGAIIGLMVGGVVIA-OH

wherein X is

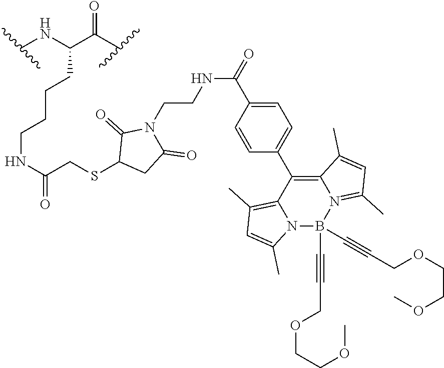

In one aspect the compound of Formula I is a compound of formula Ia, wherein formula Ia is compound of Example 3b described herein:

H-DAEFRHDSGYEVHHQ-X-LVFFAEDVGSNKGAIIGLMVGGVV-OH

wherein X is:

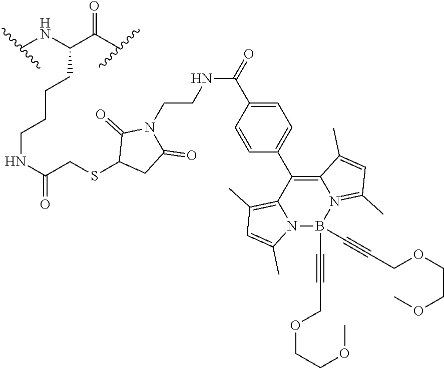

In yet another aspect, the compound of Formula I is a compound of formula Ia, wherein formula Ia is a compound of Example 3b described herein:

H-DAEFRHDSGYEVHHQ-X-LVFFAEDVGSNKGAIIGLMVGG-OH

wherein X is:

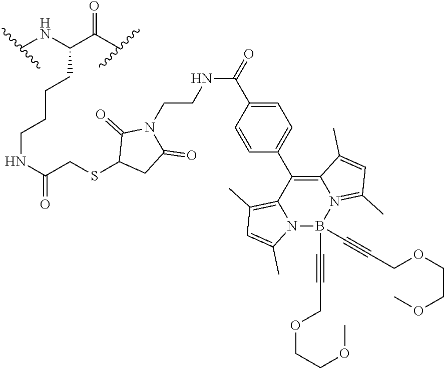

In still a further aspect, the compound of Formula I is a compound of formula Ib, wherein formula Ib is a compound of Example 9 described herein:

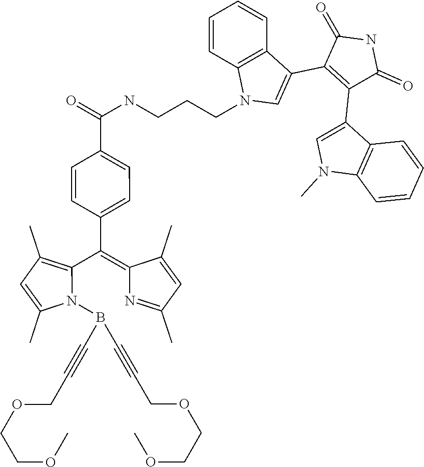 [SMILES: COCCOCC#CB(C#CCOCCOC)n1c(C)cc(C)c1/C(=C1\N=C(C)C=C1C)c1ccc(C(=O)NCCCn2cc(C3=C(c4cn(C)c5ccccc45)C(=O)NC3=O)c3ccccc32)cc1]

### II. Methods of Manufacture or Production

Compounds of the invention as described herein (e.g., compounds of Formula Ia and/or Ib and/or Ic) may be made or produced as described in this section as well as described in the Examples.

The compound of formula Ia may be prepared by reacting a compound of formula II

P—Ar—R—X′ (II)

with a compound of formula III

Tₐ-SH (III)

wherein Tₐ, P, Ar and R are each as defined above, and
X′ is amoietybearing chemical functions capable of binding covalently with the thiol function of compound of formula III and forming the spacer Xlinked through —S— to the peptidic residue Tₐ. Examples of chemical functions present in X′ include a double bond, e.g. as present in a divalent maleimido group of formula

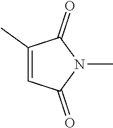

—R—X′ may also be e.g. a group chloroacetamido or chloroacetamidomethyl.

Compounds of formula II may be prepared in analogous manner as disclosed in following Examples.

Compounds of formula III may be prepared by e.g. solid phase peptide synthesis by assembling the amino acids in protected forms, e.g. as disclosed in the following Examples.

Compounds of formula Ib may be prepared by reacting a compound of formula

IV

P—Ar—(CH₂)_{d}—COOH (IV)

wherein P and Ar are as defined above and d is 0 or 1, or a functional derivative thereof, e.g. an ester, for example C₁₋₄ alkyl ester, or amide,
with a compound of formula X_{f}

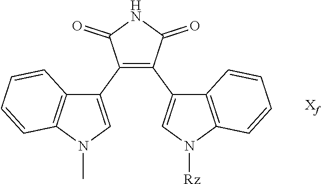

wherein R_{z} is a protecting group, cleavable to allow the further reaction with a compound of formula IV.R_{z} may be for example a phthalimidoyl group.

The above reaction may be carried out according to known procedures, e.g. as disclosed in Example 9.

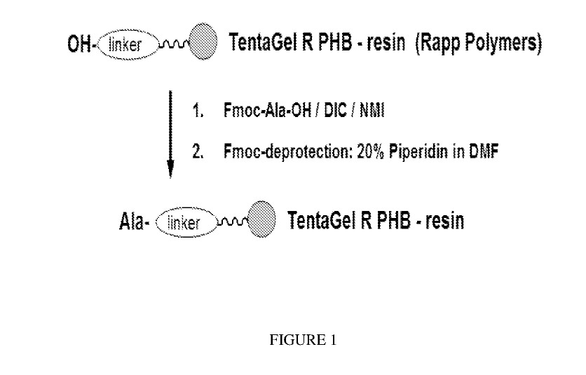

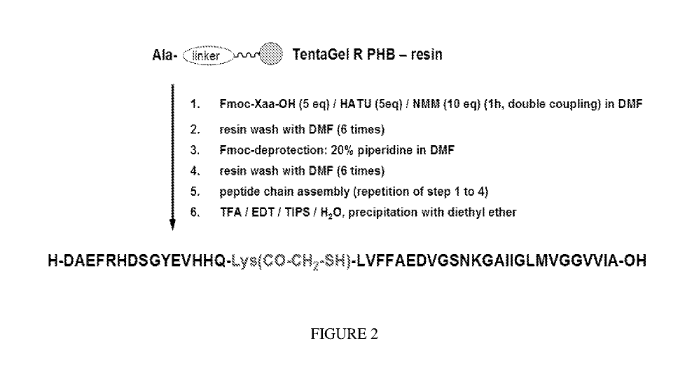

Compounds of formula IV may be prepared as disclosed in U.S. Pat. No. 8,993,781, e.g., FIGS. 1 and 2 thereof, the contents thereof being incorporated herein by reference in their entireties.

### III. Methods of Detection, Methods of Treatment, Methods of Diagnosis and Methods of Use

Compounds of Formula I (e.g., Compounds of formula Ia and/or Ib) are useful as biomarkers for the detection of neurodegenerative diseases, particularly progressive neurodegenerative diseases, e.g. Alzheimer disease (AD). Compounds of Formula I (e.g., any of formula Ia and/or formula Ib) may be used as single compound or in combination. For example, preferred combinations are a combination or an association of a compound of formula Ia with a compound of formula Ib or a compound of formula Ia with another compound of formula Ia.

Accordingly, in one aspect the invention is directed to a new biomarker and an assay with this biomarker using samples that can be collected simply, safely, and non-invasively, for example, by collecting a sample of circulating or peripherally available cells, like blood cells.

The present invention includes a complex comprising a compound of Formula I (e.g., any of Formula Ia, Ib, or Ic) and a cell or cellular membrane expressing or containing the targeted endogenous biomarker Aβ and/or PKC and related molecular signaling pathways, as a sensitive and specific biomarker of AD. This complex can be detected on peripheral cells such as red blood cells, white blood cells (monocytes, neutrophils, lymphocytes, and/or lymphocytes subtypes T and/or B), platelets, epithelial cells or stem cells, or cells of the nervous system such as cortical or hippocampal neurons or immortalized cells from patients with a neurodegenerative disease in particular AD. This marker is also detectable and measurable in cellular and animal models of AD. Since the invention shows that this cellular parameter is directly linked to the physiopathology of the disease and reports on disease progression, the present invention describes its application in diagnostic purpose, in treatment monitoring, in preclinical and clinical drug development and in developing new therapeutic pathways. This invention also concerns the methods and the kits for the detection of this biomarker.

The complex according to the invention may be obtained by incubation of said cell or cell membrane with 0.0001 to 3 μmolar of a compound of Formula I, e.g. a compound of formula Ia or Ib, in a biological fluid or an iso-osmotic medium, e.g. at a temperature between 4 and 42° C., preferentially at room temperature (RT) in case of living cells.

Use of such a complex to detect AD or a disease or disorder associated with β-amyloid deposition and/or tau hyperphosphorylation and/or diseases characterized by PKC-linked proteinopathies are also contemplated.

Another object of the present invention relates to the use of said complex for screening or profiling an agent or a drug for its use as a therapeutic agent for AD or a disease or disorder characterized by β-amyloid deposits and/or tau hyperphosphorylation and/or diseases characterized by a proteinopathy implicating PKC changes.

The cell or cell membrane component of the complex may comprise an animal cell, such as that of a human or non-human mammal, such as a mouse or rat or a non-mammal cell such as a cell from invertebrate. In some embodiments, an anucleated cell is used, such as mature human red blood cell or platelet. The membrane component of the complex may also be a cellular ghost, a liposome, a synthetic cell, or a synthetic membrane. Other kinds of cells that contain nuclei may also participate in complex formation, such as white blood cells including granulocytes, lymphocytes and monocytes, or other buffy coat cells, stem cells or cells obtained or derived from the nervous system, such as cell types present in CSF, or cells obtained or derived from the endocrine system, or cells from epithelial tissue or immortalized cell lines from patients with neurodegenerative disease, in particular AD. Other cells including artificial or modified cells, or their membranes, used to model neurological development, differentiation, or disease phenomena may be used to form complexes with the compounds of formula I (e.g., formula Ia or formula Ib) (e.g., a compound of Ex. 1, Ex. 3a, 3b, and/or 9). Preferably cells are blood cells from human individuals and they are used to form complexes with the compounds of formula I.

Complexes may also include those where the compound of Formula I, e.g. a compound of formula Ia or Ib (e.g., a compound of Ex. 1, Ex. 3a, 3b, and/or 9), is bound to the selected cell via its molecular biomarker candidate, Aβ and PKC respectively, and transiently changes the intracellular concentration of calcium and/or other second messengers in the circulating or peripheral cells of a subject.

These complexes may be formed by contacting a cell or cell membrane with a compound of formula I, e.g. a compound of formula Ia or Ib, for a time and under conditions sufficient to prime the membrane of the cell. Membrane priming can be performed and detected by exposing the cell to a 0.0001 to 3 μM concentration of a compound of formula Ia that changes (enhances or lowers depending if an Aβ-aggregating or disaggregating) peptide) or a compound of formula Ib that increases or decreases (depending if positive or negative modulator of PKC) the level of intracellular calcium ion concentration in a primed cell to a level different (higher or lower) than that measured in unprimed cell not previously contacted with a compound of formula I. Since the intracellular calcium concentration triggers metabolic cascades in intact cells, it is also possible to detect membrane priming by measuring cellular parameters triggered by calcium such as protein conformation like protein kinase C (PKC), enzymatic activities, calcium triggered ionic channels or changes in the downstream intracellular targets and pathways.

In another aspect, the invention is directed to a composition comprising at least one of:
one or more reagents for isolating or purifying a cell or a cell membrane to which a compound of formula I has or will be bound;
one or more reagents for incubating the cells or cell membranes with a compound of formula I, e.g. a compound of formula Ia or Ib;
one or more reagents for enhancing and/or inhibiting the binding and/or interaction of a compound of formula I with a cell or cell membrane;
one or more reagents for measuring a complex of a compound of formula I with the cell or cell membrane;
and to a kit, comprising a composition as disclosed above and herein (e.g., a compound of Formula I), and
a device to detect or quantify the amount of complex formation; and/or
a software for detecting, quantifying or otherwise analyzing complex formation, and/or
written instructions or user manual for using the kit to detect or assess the risk of AD.

Examples of reagents which may be present in the composition or kit to enhance and/or inhibit the binding and/or interaction of a compound of formula Ia or Ib with a cell or cell membrane are a compound of formula Ia or Ib, or one of (but not limited to) known molecules e.g. a PKC activator or inhibitor, including but not limited to a phorbol ester, for example phorbol-12-myristate-13-acetate (PMA), staurosporine, a PKC activating or inhibiting agent, or an antibody against APP or against Aβ1-42 or against one of the shorter fragment of Aβ1-42, or a molecule binding to and aggregating with Aβ1-42 or one its shorter peptides, or a molecule binding to and disaggregating Aβ1-42 or its shorter peptides, or several of these reagents. The composition or kit of the invention may also comprise a buffer, e.g. a buffered iso-osmotic solution containing 2-[4-(2hydroxyethyl)piperazin-1-yl]ethanesulfonic acid or trishydroxymethylamino-methane or phosphatidic acid or trifluoroacetic acid or ethanol or dimethylsulfoxyde or hexafluoro-isopropanol or several of these compounds.

Yet another aspect of the invention is a method for detecting the presence of a compound of Formula I—cell membrane complex comprising:
a) purifying or isolating cells or cell membranes from a biological sample (e.g., erythrocytes) of a subject suspected of having, or at risk of developing AD or a disease characterized by deposits of β-amyloid peptides in brain or nervous system;
b) contacting the purified or isolated cells or cell membranes with a compound of formula I, e.g. a compound of formula Ia or Ib ((e.g., a compound of Ex. 1, Ex. 3a, Ex. 3b, or Ex. 9); preferably at a concentration between 0.0001 and 3 μM for a time and under conditions suitable for complex formation; preferably in an iso-osmotic medium and/or at a temperature between 4 and 42° C. and/or during an incubation time of from 5 minutes to 3 hours; preferably at room temperature for living blood cells and other living cells;
c) enhancing and/or inhibiting the binding and/or interaction of a compound of formula I, e.g. a compound of formula Ia or Ib ((e.g., a compound of Ex. 1, Ex. 3a, Ex. 3b, or Ex. 9)), with a cell or cell membrane, e.g. by addition of an appropriate agent;
d) detecting complex formation between the cells or cell membranes and the compound of formula I, preferably by measuring the fluorescence (e.g. using flow cytometry) of compound of formula I, e.g. a compound of formula Ia or Ib ((e.g., a compound of Ex. 1, Ex. 3a, Ex. 3b, or Ex. 9)) bound to said cells or cell membranes.

The method above, wherein steps a)-d) utilize a compound e.g., Formula Ia and/or Ib (e.g., a compound of Ex. 1, Ex. 3a, Ex. 3b, or Ex. 9).

The method may comprise a further step comparing the amount of complex formation to the amount of complex formation in a normal subject, in a subject not having AD, or to a normal control value, and diagnosing the subject as having AD or as being at risk of developing AD when complex formation is higher or lower according to the test compound bound to respective biomarker than that in the normal subject, in a subject not having AD, or in a normal control.

In one aspect, the invention is directed to method for diagnosing a subject as having AD or for being at risk of developing or progressing for AD or a disease or disorder characterized by the abnormal deposition of β-amyloid and/or tau and/or abnormal PKC changes and/or other proteins downstream to PKC and/or β-amyloid changes comprising:
a) purifying or isolating cells or cell membranes from a biological sample (e.g., blood sample) of a subject suspected of having, or at risk of developing AD or a disease characterized by deposits of β-amyloid peptides in brain or nervous system;
b) contacting the purified or isolated cells or cell membranes with a compound of Formula I, e.g. a compound of formula Ia or Ib (e.g., a compound of Ex. 1, Ex. 3a, Ex. 3b, or Ex. 9);
c) enhancing and/or inhibiting the binding and/or interaction of a compound of formula I, e.g. a compound of formula Ia or Ib (e.g., a compound of Ex. 1, Ex. 3a, Ex. 3b, or Ex. 9), with a cell or cell membrane, e.g. by addition of an appropriate agent;
d) detecting complex formation between the cells or cell membranes and the compound of formula I ((e.g. a compound of formula Ia or Ib)(e.g., a compound of Ex. 1, Ex. 3a, Ex. 3b, or Ex. 9)), preferably by measuring the fluorescence of compound of formula I, bound to said cells or cell membranes;
e) comparing the amount of complex formation to a reference standard (e.g., normal subject, in a subject not having AD, or to a normal control value), and;
f) diagnosing whether the subject has AD or is being at risk of developing AD when complex formation is higher or lower according to the test compound bound to respective biomarker compared to the reference standard.

The method above, wherein steps a)-d) utilize a compound e.g., Formula Ia and/or Ib (e.g., a compound of Ex. 1, Ex. 3a, Ex. 3b, or Ex. 9).

The above method can be conducted where the blood samples are erythrocytes and flow cytometry is used to determine staining/fluorescence.

The method may also comprise a further step comparing the amount of complex formation between the cell or cell membrane and a compound of formula I to the amount of complex formation in a normal subject, in a subject not having a disease or disorder characterized by β-amyloid deposit or tau deposit or proteinopathy implicating abnormal PKC change, or to a normal control value, and diagnosing the subject as having such a disease or disorder or as being at risk of developing such a disease or disorder when complex formation is higher than that in the normal subject, in a subject not having such a disease or disorder, or in a normal control.

The above described method may use a mammalian cell or cell membrane, such as a human, primate, rat or murine cell. For example, the method may be practiced on cells from human or animal or from an animal modeling human AD or from a human patient with AD or other disease involving abnormal or pathological deposition of β-amyloid and/or tau and/or pathological changes implicating PKC protein and/or activity.

Preferably when using a compound of formula Ib, as when using a compound of formula Ia, the cells are red or white blood cells or membranes thereof.

The cell or membrane may be primed as described above with a compound of formula I, preferably at a concentration between 0.0001 and 3 μM; and wherein membrane priming is performed and detected by exposing the cell preferably to a 0.001 to 3 μM concentration of compound of formula I.

In the methods above, the subject may be human, primate, a non-primate mammal, or other non-human animal. Transgenic animal models of AD expressing mutant genes implicated in familial forms of AD may be used. The methods above may also purify or isolate cells or cell membranes of such a subject by collecting peripheral cells such as epithelial cells or cells associated with or forming a mucous membrane, or the circulating cells of the subject, such as blood cells. Cells isolated or derived from the nervous system of the membranes of such cells maybe used. Preferably, peripheral cells not requesting invasive sample collection are preferred, in particular blood cells including red blood cells.

The AD assessment and assays of the invention may be performed on one sample or on longitudinally collected samples from the same subject or from a cohort of subjects, such as subjects being followed in longitudinal studies aiming at understanding the mechanisms of the conversion of MCI to AD and the non-conversion of MCI to AD, or subjects being treated for AD or for a disease or disorder characterized by the abnormal or pathological deposit of amyloid plaques, neurofibrillary tangles and/or other proteinopathy characterized by abnormal changes in PKC.

A further aspect of the invention involves a method for diagnosing a subject as having AD or for being at risk of developing or progressing for AD or a disease or disorder characterized by the abnormal deposition of β-amyloid and/or tau and/or abnormal PKC changes and/or other proteins downstream to PKC and/or β-amyloid changes comprising the steps of:
a) purifying or isolating cells or cell membranes from a biological sample (e.g., a blood sample; e.g., erythrocytes) of a subject suspected of having, or at risk of developing or progressing to, AD or such a disease or disorder,
b) contacting the purified or isolated cells or cell membranes with a compound of formula Ib (e.g., compound of Ex. 9), e.g. under conditions including an iso-osmotic medium temperature between 4 and 42° C. and an exposure time, which allows said compound to be bound to cell membranes and/or loaded inside the cells,
c) contacting the purified or isolated cells with a compound of formula Ia (e.g., a compound of Ex. 1, Ex. 3a, and/or Ex. 3b) at a concentration between 0.0001 and 3 μM for a time and under conditions sufficient for the compound of formula Ia to enable sufficient detectable binding signal, preferably said conditions include e.g. an iso-osmotic medium and a temperature between 4 and 42° C., and detecting the fluorescent staining of the cells contacted with the compound of formula Ia and formula Ib,
e) comparing the fluorescent staining level (e.g., using flow cytometry) of the cells from a patient with AD or another degenerative disease or disorder to the fluorescent staining level of the cells from a normal subject, a subject not having AD, or to a normal control value, or to the fluorescent staining of a positive or a negative calibrator in a stabilized blood sample,
f) diagnosing the subject as having AD or other degenerative disease or disorder or as being at risk of developing or progressing for AD or other degenerative disease or disorder when the fluorescent staining level of cells determined for a patient with AD or other degenerative disease or disorder is higher than the fluorescent staining level of cells determined for the normal subject, a subject not having AD, or other neurodegenerative disease or disorder, or normal control value.

One way to measure the fluorescent staining is by flow cytometry or by means of a microscopy device equipped with a spectral detector.

In step d) the detection of complex formation between the cells and the compound of formula Ia and/or the compound of formula Ib may be performed by measuring the fluorescence bound to said cells using additional fluorescence reading methods, such as spectrofluorometry.

The above method may also be performed by measuring two or more fluorescent staining obtained with two or more fluorescent compounds of formula I using one of the techniques such as flow cytometry or spectrofluorometry or by means of a microscopy device equipped with a spectral detector, and:
a) the same cell population type from the same subject, e.g. measuring in parallel the staining of erythrocytes with a compound of formula Ia and the staining of erythrocytes with a compound of formula Ib; or
b) different cell population types from the same subject, e.g. measuring the staining of erythrocytes by a compound of formula Ia and the staining of lymphocytes B by a compound of formula Ib.

The above detection and diagnosis methods may be further performed using a software, an algorithm, statistical and/or another integrative tool. Such tools may also enable the combination and/or the correlation of the scores of the fluorescent staining according to the invention, with the scores of existing diagnosis biomarkers of the same subject such as neuroimaging scores, CSF biomarkers scores and/or neuropsychological scores.

In another aspect, the invention pertains to a method for screening an agent or drug that changes the cell staining by a compound of Formula I, e.g. a compound of formula Ia or Ib, for use as a therapeutic agent for AD, or for a disease or disorder characterized by β-amyloid and/or tau deposits and/or abnormal changes in PKC, comprising:
a) purifying or isolating cells or cell membranes,
b) contacting the isolated cells or cell membranes (i) with an agent or drug to be tested alone, (ii) with the excipient or vehicle of this agent or drug alone, (iii) with 0.0001 to 3 μM of a compound of formula I alone, and (iv) with the agent or drug to be tested, in the presence of 0.0001 to 3 μM of a compound of formula I under appropriate conditions that include an iso-osmotic medium, a temperature of from 4 to 42° C. and an incubation time of from 5 minutes to 24 hours,
c) detecting background or baseline values (only vehicle or excipient alone, only agent or drug alone) and detecting complex formation between the cells or cell membranes and the compound of formula I by measuring said compound of formula I bound to said cells or cell membranes in the two conditions that are in the absence or the presence of the test agent or drug,
d) subtracting baseline or background values and comparing the resulting amount of complex formation in the cells contacted with the agent or drug and with said compound of formula I to that obtained with said compound of formula I alone,
e) selecting an agent or a drug that changes the complex formation.

In steps c), d) and e): in addition to measurement of the amount of complex (compound of formula I bound to said cells or cell membranes), the parameter of a signaling cascade triggered by the compound of formula I and/or modulated the tested agent or drug (changes in intracellular calcium, other second messengers downstream to Aβ or PKC, e.g. diacylglycerol (DAG)) are also measured.

An associated embodiment is an agent or a drug identified by the methods above, the use of the agent or drug to treat a disease or disorder, such as AD, characterized or associated with deposition of β-amyloid and/or or tau hyperphosphorylation and/or associated with proteinopathy characterized by PKC-related abnormalities. The agent or drug may be used for treating AD or a disease or disorder characterized by β-amyloid and/or tau deposition and/or abnormal changes in PKC. Generally, this comprises administering an effective amount of the agent or drug identified by the methods above to a subject in need thereof.

Within these methods, the cells or cell membranes can be of human origin, of non-human animal origin, or can be cultured cells, which may be primary cultured cells or cells from a cultured cell line or immortalized cell line from patients.

In another aspect, the invention relates to a method for screening an agent that changes the cell staining by a compound of formula I, e.g. a compound of formula Ia or Ib, for use as a new agent for diagnosis of AD or a disease or disorder characterized by β-amyloid and/or tau deposits and/or abnormal changes in PKC, comprising the similar steps as those described above in the invention for screening an agent or drug for use as therapeutic agent, except that the therapeutic agent or drug is replaced by the novel screened diagnosis agent.

Another aspect of the invention pertains to the use of the compounds of the invention for in vitro histological staining of human postmortem tissue such as brain or as a histological method for in vitro or in vivo staining of animal tissue, such as brain tissue, for additional applications of diagnosis, screening, discovery and development of test agents or drugs. Compounds of formula Ia and Ib are useful in a method for in vitro and in vivo histological fluorescence staining; compounds of formula Ia are preferably useful in a method for in vivo neuroimaging PET ligand to visualize brain amyloidosis and compounds of formula Ib to visualize PKC abnormalities in the brain of subjects for diagnosis purposes and for drug development applications.

The in vitro method of staining of postmortem tissue, in particular brain tissue, with a compound of formula I comprises the following main steps:
a) fixing brain tissue blocks in a buffer containing a fixative agent, preferably in an amount of 0.1-4%
b) preparing transverse or sagittal brain sections, e.g. using a vibratome or cryostat or preparation of paraffin sections,
c) incubating the brain sections with the buffer alone (for baseline) and with 0.01 to 3 μM compound of formula Ia and/or Ib in the buffer,
d) washing the brain sections in the buffer, and
e) analyzing the fluorescence of the stained tissue section, e.g. using a confocal or other fluorescence microscopy equipped with an imagery system for quantification of staining and/or counting stained cells, and
f) determining whether the tested agent or drug reduces or enhances the pathological lesions stained with said compound of formula I.

The in vivo method of staining of tissue (e.g. brain tissue) with a compound of formula I comprises the following main steps:
a) injecting intravenously 0.01 to 10 mg/kg of a compound of formula I in animals,
b) sacrificing the treated animals 15 min to 6h later,
c) removing the brain and fixing as described above,
d) preparing the sections as described above and analyzing the fluorescence, e.g. directly by fluorescence microscopy to visualize cells and the pathological lesions stained with the compound of formula I.

The implementation of this detection method with the compounds of the invention allows direct enabling without use of other tools to visualize the brain areas stained as well as cell types stained: in particular, the staining with the compounds of formula Ia, of extracellular deposits and amyloid plaques in the parenchyma of the postmortem human brain and brain of transgenic animal models of AD, and the staining with the compounds of formula Ib of brain neuronal cells expressing PKC. Animals models used can also be those overexpressing or deficient in PKC or in a molecular target upstream or downstream in the PKC or to APP or tau pathways.

The above in vitro and in vivo methods of staining of brain tissue with a compound of Formula I can also be used for drug screening and development in animal models. Accordingly, this method comprises:
a) Treatment with tested drug or agent is performed before in vitro staining of tissue section with a compound of formula I, e.g. groups of transgenic animals with brain amyloidosis or tauopathy are first treated acutely or chronically with the test agent or drug in parallel to one group treated with the vehicle of tested agent or drug. The animals are then sacrificed and the brain removed, fixed and brain sections prepared and stained as described above before microscopically analysis to determine whether the tested agent or drug reduces or enhances the pathological lesions stained with a compound of formula I.
b) Treatment with tested drug or agent is performed before an in-vivo staining of tissue section with compound of formula I, e.g. after treatment of the animals with the test agent or drug as disclosed above, the animals are given an intravenous injection of 0.01 to 10 mg/kg of a compound of formula I, and then sacrificed 15 min to 6h later, the brain removed and fixed, and the sections prepared and directly analyzed by fluorescence microscopy to determine whether the test agent or drug reduces the pathological lesions stained with the compound of formula I.

The in vivo method for staining brain tissue with a compound of formula I is also an important step to the process of developing a compound of formula Ia or Ib as a neuroimaging ligand: it enables to screen and select the best compound of formula Ia and of formula Ib that a) crosses the brain, b) reaches and stains its biomarkers containing cells and/or lesions in the brain, c) the resulting staining showing specificity versus the background.

Another aspect of the invention involves a method, (e.g., a non-invasive method), for detecting alterations in a cell or a cell membrane of a circulating or peripheral cell induced by AD or a disease or disorder characterized by deposition of β-amyloid and/or tau and/or by PKC-related abnormalities comprising:
a) purifying or isolating cells or cell membranes from a biological sample of a subject suspected of having, or at risk of developing, AD or a disease characterized by deposits of β-amyloid peptide, and/or tau hyperphosphorylation and/or associated with proteinopathy characterized by PKC-related abnormalities,
b) contacting the purified or isolated cells or cell membranes with a compound of Formula I, e.g. a compound of formula Ia or Ib (a compound of Ex. 1, Ex. 3a, 3b, or Ex. 9), at a concentration-between 0.01 and 3 μmolar for a-time and under conditions suitable for complex formation, in the presence or the absence of reagent(s) for enhancing and/or inhibiting the binding and/or interaction of a compound of formula I with a cell or cell membrane, preferably said conditions including an iso-osmotic medium and a temperature between 4 and 42° C.,
c) detecting complex formation between the cells or cell membranes and the compound of formula I by measuring the fluorescence bound to said cells or cell membranes; and
d) measuring in said isolated cells or cell membranes obtained in step b) a change in the AP or its downstream molecular cascade, such as aggregation, binding, calcium changes and calcium related pathways, or changes in PKC conformation or activity or expression or at least one other parameter associated with AD or a disease or disorder characterized by deposition of β-amyloid, and/or tau hyperphosphorylation and/or associated with proteinopathy characterized by PKC-related abnormalities (e.g. ERK1/2 activity, GSK-3-beta activity that impact Tau hyperphosphorylation).

The reagent(s) for enhancing and/or inhibiting the binding and/or interaction of a compound of formula I with a cell or cell membrane are as indicated above.

Within the methods described herein, the cells or cell membranes can be obtained from a human subject who has AD or who is at risk of developing AD, from a non-human animal subject having or at risk of developing a disease or disorder characterized by β-amyloid deposition similar to human AD, from an animal that models human AD, or from cultured, modified or artificial cells. This method may be employed to detect, diagnose, or evaluate a human for AD or to detect, diagnose or evaluate a non-human animal or transgenic animal model of human AD.

Another embodiment represents a method for diagnosing a subject as having a disease or disorder associated with or characterized by the deposit of β-amyloid and/or tau or by abnormal changes in PKC, such as AD, comprising noninvasively isolating a circulating cell or a peripheral cell of a subject, detecting an alteration in the membrane of said cell compared to a normal cell, and diagnosing the subject as having said disease or disorder when the membrane of the cell is altered compared to the membrane of a normal subject not having said disease or disorder. This method may isolate or purify an anucleated cell for noninvasive testing, such a red blood cell from the peripheral circulation or an epithelial or a cell associated with a mucous membrane, according to the invention.

Detection of the primed binding on cells or on a cellular membrane preparation, as described herein, may be performed by fluorescence measurement, colorimetry, flow cytometry, immunochemistry or immunofluorescence, optionally radioactivity, NMR, PET, EPR.

Another embodiment of the invention concerns the simultaneous or sequential measurement in vitro of the peripheral biomarker(s) of the present invention (related to Aβ using a compound of formula Ia or related to PKC and measured using a compound of formula Ib), with another biomarker besides those described here including and not limited to the combinations of the biomarkers of the present invention with the in vitro CSF tests (Aβ, tau and/or phospho-tau) and/or blood biomarkers detected by antibodies, and/or RNA expression and/or genotypes measured in a peripheral body fluid sample, such as APOE alleles, in particular allele APOE E4 for diagnosis, therapeutic treatment monitoring or drug development purposes.

Another embodiment of the invention concerns the simultaneous or sequential measurement of the peripheral biomarker(s) of the present invention (related to Aβ using a fluorescent compound of formula Ia (e.g., Ex. 1, Ex. 3a, or Ex. 3b) or related to PKC and measured using a fluorescent compound of formula Ib (e.g., Ex. 9)) measured in vitro, with an in vivo test including and not limited to the combinations of the biomarkers of the present invention measured with the in vitro method of the invention in the peripheral cells with neuroimaging (such as structural magnetic resonance imaging (MRI) or Positron emission tomography (PET) using one of the existing radiotracers monitoring brain metabolism such as ¹⁸F-fluorodeoxyglucose or radiotracers of brain amyloidosis such as ¹⁸F-florbetapir and ¹⁸F-flutemetamol),and/or neuropsychological scores (such as Mini Mental State Examination, Free and cued selective reminding test, Montreal cognitive assessment, Rey auditory verbal learning test, Clock drawing test, AD assessment Scale-cognitive subscale, trail making test, Functional Activities Questionnaire, or others tests) for diagnosis, therapeutic treatment monitoring or drug development purposes.

Another embodiment of the invention concerns the simultaneous or sequential measurement of the peripheral biomarker(s) of the present invention (related to Aβ using a fluorescent compound of formula Ia (e.g., Ex. 1, Ex. 3a, or Ex. 3b) or related to PKC and measured using a fluorescent compound of formula Ib (e.g., Ex. 9)) measured in vitro in peripheral cells, particularly blood cells, preceding, replacing, limiting or avoiding the use of the existing invasive methods (such as CSF biomarkers that require lumbar puncture), both for diagnosis purposes and for applications in drug development in clinical trials.

Another embodiment of the invention concerns the simultaneous or sequential measurement of the peripheral biomarker(s) of the present invention (related to Aβ using a fluorescent compound of formula Ia or related to PKC and measured using a fluorescent compound of formula Ib) measured in vitro in peripheral cells, particularly blood cells, for population screening and for applications related to implementing preventive strategies.

The following examples illustrate the invention without any limitation. In the following Examples, Bodipy is P—Ar— wherein P is a residue of formula (a), wherein each of R₁, R₃, R₄ and R₆ is CH₃, each of R₂ and R₅ is H, and each of S₁ and S₂ is

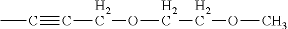

and Ar is 1,4-phenylene.
Rt=room temperature

### Example 1: Lys-[N-2-(3-CO—CH₂—S-2,5-dioxo-pyrrolidinyl)-ethylamido-Bodipy]-Beta-Amyloid (1-42)

H-DAEFRHDSGYEVHHQ-X-LVFFAEDVGSNKGAIIGLMVGGVVIA-OH

wherein X is

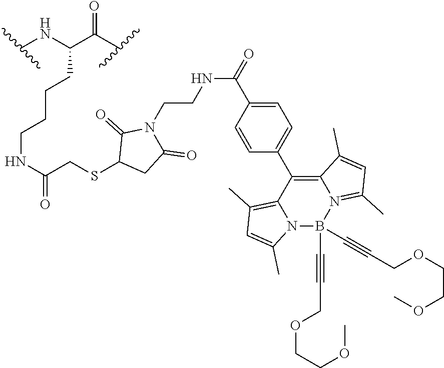

a) H-Ala-TentaGel R PHB resin
Synthesis of Alanine-loadedTentaGel R PHB resin is performed using a standard procedure for resin loading as indicated by the procedure in FIG. 1.
The resulting amino acid loading of the resin is 0.16 mmol/g

### b) Synthesis of Fmoc-Lys(CO—CH₂—S-Trt)-OH

This building block is needed for the site specific incorporation of a mercaptoacetic acid modified lysine derivative during solid phase peptide synthesis and is synthesized according to the following procedure.

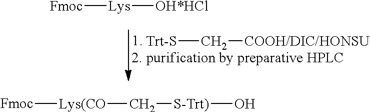

HONSU: N-Hydroxysuccinimide

Trt-S—CH₂—COOH:Trityl-protected Mercaptoacetic acid

Purification is performed by preparative HPLC (Dionex) using a PLRP-S column (300*50 mm). For purification a 80 min-gradient of 30-80% acetonitrile (ACN) in water is used (flow: 40 ml/min). Detection is performed at 220 nm. After lyophilization, the building block is stored at −20° C.
purity: 99.2% Mₜₕₑₒ: 684.85M_{found}: 684.01
c) Solid phase peptide synthesis (peptide chain assembly): Lys-(CO—CH₂—SH)-beta-Amyloid (1-42)
For peptide synthesis the following amino acid derivatives are used:
Fmoc-Ile-OH, Fmoc-Val-OH, Fmoc-Gly-OH, Fmoc-Met-OH, Fmoc-Leu-OH, Fmoc-Ile-OH, Fmoc-Ala-OH, Fmoc-Lys(Boc)-OH, Fmoc-Asn(Trt)-OH, Fmoc-Ser(tBu)-OH, Fmoc-Asp(OtBu)-OH, Fmoc-Glu(OtBu)-OH, Fmoc-Phe-OH, Fmoc-Lys(CO—CH₂—S-Trt)-OH, Fmoc-Gln(Trt)-OH, Fmoc-His(Trt)-OH, Fmoc-Tyr(tBu)-OH, Fmoc-Asp(OtBu)-OH, Fmoc-Arg(Pbf)-OH, Peptide synthesis is performed using an ABI synthesizer of Applied Biosystemsaccording to the procedure detailed in FIG. 2 (synthesis scale: 0.32 mmol).
d) Purification of the crude peptide

Purification is performed by preparative HPLC (Dionex) using a PLRP-S column (300*50 mm). As solvents ACN (0.1% TFA) and water (0.1% TFA) are used. For purification a gradient of 10-95% ACN in 80 min is used (flow: 40 ml/min). Detection is performed at 220 nm. After lyophilization, the peptide is stored at −20° C.

Mₜₕₑₒ: 4588.17M_{found}: 4588.8
e) Synthesis of Bodipy-CO—NH—(CH₂)₂-Maleimide

Synthesis of Bodipy-CO—NH—CH₂—CH₂-Maleimide is performed according to the following picture. The starting material Bodipy-CO—OH (206 mg) is produced as disclosed in U.S. Pat. No. 8,993,781B2 (Compound 3a, starting from compound 1a and following the procedure indicated in FIG. 2)

Bodipy-CO—OH [symbol] Bodipy-CO—NH—CH₂—CH₂-maleimide
1. DIC/HONSU
2. N-(2-Aminoethyl)maleimide hydrochloride/NMM
   Purification is performed by preparative HPLC (Dionex) using a C18 column (250*21 mm). As solvents ACN (0.1% TFA) and water (0.1% TFA) are used. For purification a gradient of 0-90% ACN in 120 min is used (flow: 12 ml/min). Detection is performed at 220 nm. After lyophilization, the compound is stored at −20° C.
   Mₜₕₑₒ: 678.39M_{found}: 678.3
a) Lys4N-2-(3-CO—CH₂—S-2,5-dioxo-pyrrolidinyl)-ethylamido-Bodipyd-Beta-Amyloid (1-42)

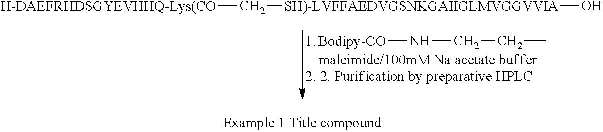

Purification is performed by preparative HPLC (Dionex) using a C18 column (250*19 mm). As solvents ACN (0.1% TFA) and water (0.1% TFA) are used. For purification a gradient of 20-90% ACN in 80 min is used (flow: 10 ml/min). Detection is performed at 220 nm. Afterlyophilization, the compound is stored at −20° C.
Peptide quality is analyzed using a mass spectrometer coupled HPLC (Agilent LC-MS system Infinity 1200 with 6230-TOF mass detector) using a C18 column (50*2.1 mm). As solvents ACN (0.05% TFA) and water (0.05% TFA) are used (gradient: 5-95% ACN in 6 min, flow: 0.5 ml/min)
purity: 90.2% Mₜₕₑₒ: 5266.56M_{found}: 5266.18

### Example 2: 16-Cys-[N-2-(2,5-dioxo-pyrrolidinyl)-ethylamido-Bodipy]-Beta-Amyloid (1-42)

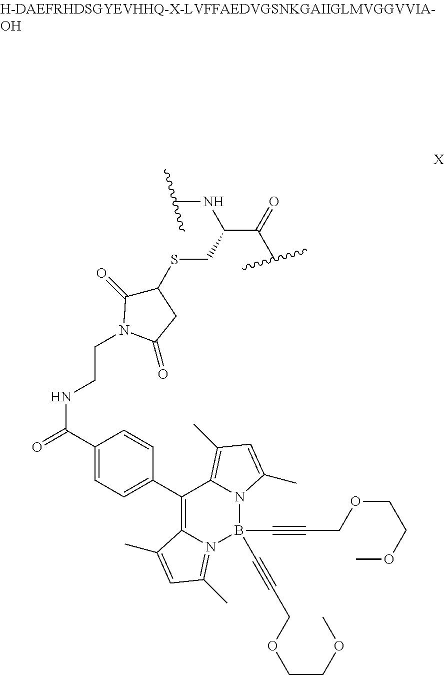

a) Solid phase peptide synthesis (peptide chain assembly): 16-Cys-Beta-Amyloid (1-42)

For peptide synthesis the following amino acid derivatives are used:

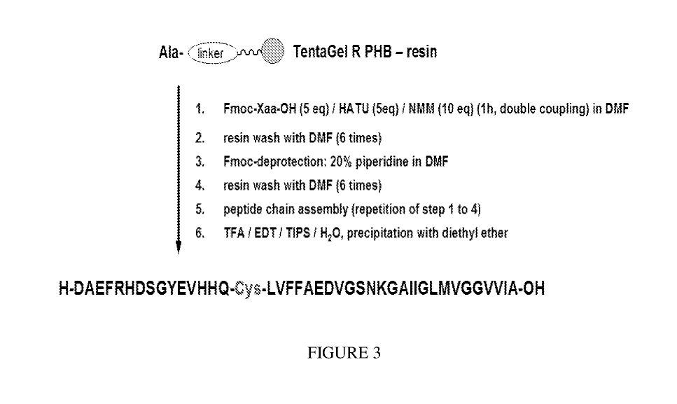

Fmoc-Ile-OH, Fmoc-Val-OH, Fmoc-Gly-OH, Fmoc-Met-OH, Fmoc-Leu-OH, Fmoc-Ile-OH, Fmoc-Ala-OH, Fmoc-Lys(Boc)-OH, Fmoc-Asn(Trt)-OH, Fmoc-Ser(tBu)-OH, Fmoc-Asp(OtBu)-OH, Fmoc-Glu(OtBu)-OH, Fmoc-Phe-OH, Fmoc-Cys(Trt)-OH, Fmoc-Gln(Trt)-OH, Fmoc-His(Trt)-OH, Fmoc-Tyr(tBu)-OH, Fmoc-Asp(OtBu)-OH, Fmoc-Arg(Pbf)-OH, Fmoc-Cys(Trt)-OH
Peptide synthesis is performed using an ABI synthesizer of Applied Biosystemsaccording to the procedure detailed in FIG. 3 (synthesis scale: 0.32 mmol).
b) Purification of the crude peptide

Purification is performed by preparative HPLC (Dionex) using a PLRP-S column (300*50 mm). As solvents ACN (0.1% TFA) and water (0.1% TFA) are used. For purification a gradient of 0-90% ACN in 80 min is used (flow: 40 ml/min). Detection is performed at 220 nm. After lyophilization, the peptide is stored at −20° C.

Mₜₕₑₒ: 4489.04 M_{found}: 4489.26
c) 16-Cys-[N-2-(2,5-dioxo-pyrrolidinyl)-ethylamido-Bodipy]-Beta-Amyloid (1-42)

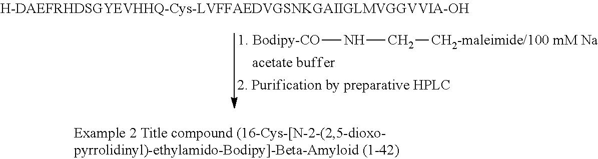

d) Purification of 16-Cys-[N-2-(2,5-dioxo-pyrrolidinyl)-ethylamido-Bodipy]-Beta-Amyloid (1-42)

Purification is performed by preparative HPLC (Dionex) using a C18 column (250*19 mm). As solvents ACN (0.1% TFA) and water (0.1% TFA) are used. For purification a gradient of 20-90% ACN in 80 min is used (flow: 10 ml/min). Detection is performed at 220 nm. Afterlyophilization, the compound is stored at −20° C.

Peptide quality is analyzed by MALDI TOF mass spectrometry using a Voyager-DE mass spectrometer of PerSeptiveBiosystems
Mₜₕₑₒ: 5167.43 M_{found}: 5167.57

### Example 3

By following the procedure of Example 1, the following compounds of formula Ia may be obtained:

| | |
|---|---|
| (a) | H-DAEFRHDSGYEVHHQ-X-LVFFAEDVGSNKGAIIGLMVG |
| | GVV-OH |
| (b) | H-DAEFRHDSGYEVHHQ-X-LVFFAEDVGSNKGAIIGLMVG |
| | G-OH |
| (c) | H-DAEFRHDSGYEVHHQ-X-OH |
| (d) | H-X-LVFFAEDVGSNKGAIIGLMVGGVVIA-OH |
| (e) | H-QSHYRHISPAQVHHQ-X-OH, |
| and | |
| (f) | H-RPRTRLHTHRNRHHQ-X-OH |

wherein X is as indicated for the compound of Example 1.

### Example 4: Chloroacetamide Modified Bodipy

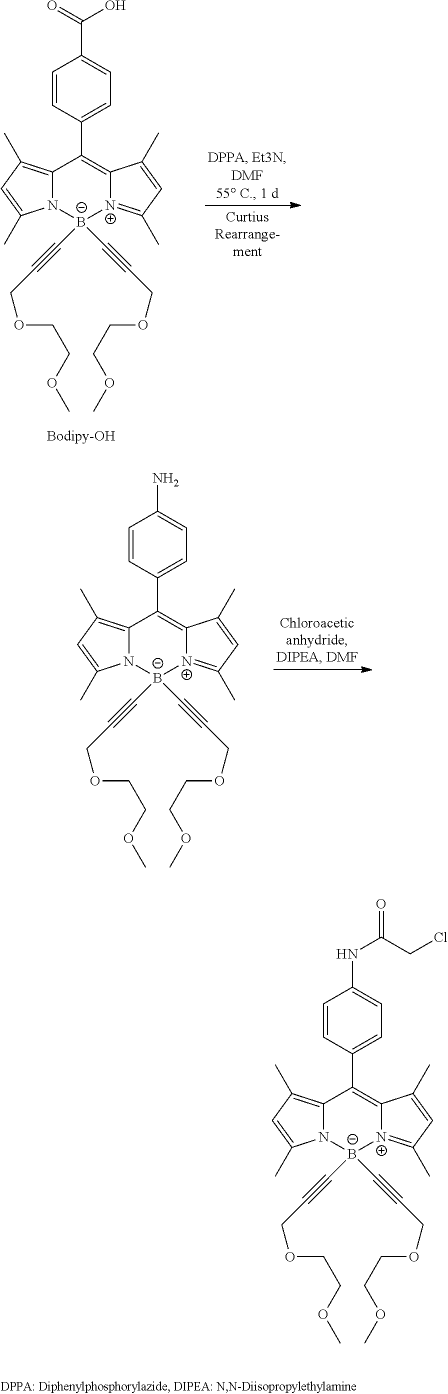 [SMILES: COCCOCC#C[B-]1(C#CCOCCOC)n2c(C)cc(C)c2C(c2ccc(C(=O)O)cc2)=C2C(C)=CC(C)=[N+]21 ; COCCOCC#C[B-]1(C#CCOCCOC)n2c(C)cc(C)c2C(c2ccc(N)cc2)=C2C(C)=CC(C)=[N+]21 ; COCCOCC#C[B-]1(C#CCOCCOC)n2c(C)cc(C)c2C(c2ccc(NC(=O)CCl)cc2)=C2C(C)=CC(C)=[N+]21]

Bodipy-CO—OH (100 mg, 180 μmol) (Ulrich et al. J. Org. Chem. 2012, 77, 5036-5048, Compound 10) is dissolved in anhydrous DMF (1.0 mL). Diphenylphosphorylazide (DPPA, 49.8 μL, 231 μmol 1.3 eq.) and triethylamine (49.9 μL, 360 μmol, 2.0 eq.) are added and the resulting solution is stirred at 55° C. for 16 hours. After cooling to RT, water (20 mL) and saturated aqueous ammonium chloride solution (20 mL) are added followed by extraction with DCM (3×30 mL). The combined organic layers are dried over Na₂SO₄, filtered and evaporated under reduced pressure. Purification by HPLC yields the desired aniline derivative.

The aniline derivative (5.0 mg, 9.5 μmol) is dissolved in a mixture of DMF (200 μL) and DIPEA (4.8 μL, 28.2 μmol, 3.0 eq.). Chloroacetic anhydride (3.2 mg, 18.7 μmol, 2.0 eq.) is added and the solution stirred at RT for 2 hours. Additional chloroacetic anhydride (1.6 mg, 9.4 μmol, 1.0 eq.) is added and the solution stirred for another 2.5 hours. Following acidification with acetic acid (30 μL) the solution is subjected to HPLC purification, thus yielding chloroacetamide modified Bodipy.

[M+H]+ₜₕₑₒ: 604.27 [M+H]+_{found}: 604.3

### Example 5: Chloroacetamidomethyl Modified Bodipy

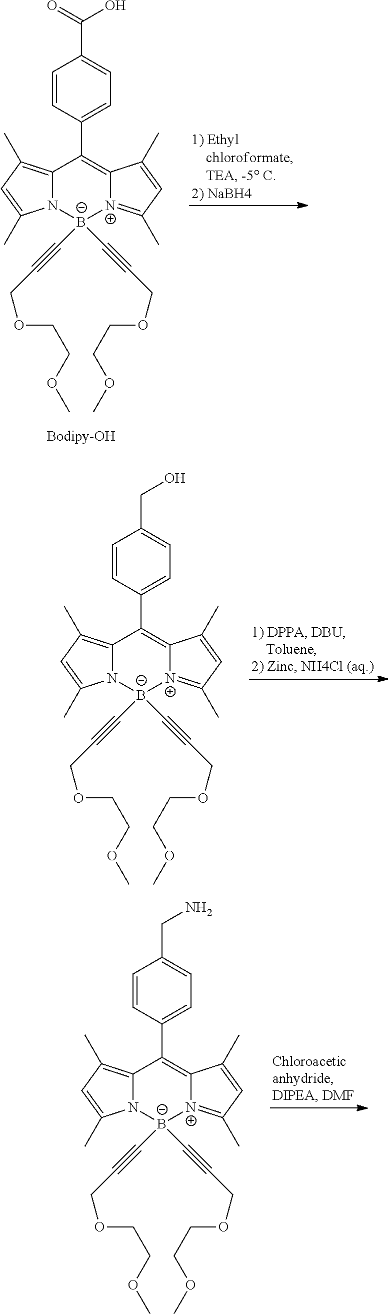

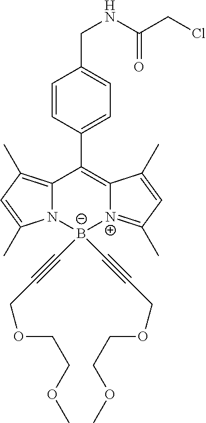 [SMILES: COCCOCC#C[B-]1(C#CCOCCOC)n2c(C)cc(C)c2C(c2ccc(CNC(=O)CCl)cc2)=C2C(C)=CC(C)=[N+]21]

In a dried round bottom flask under atmosphere Bodipy-CO—OH (450 mg, 0.81 mmol) (Ulrich et al. J. Org. Chem. 2012, 77, 5036-5048, Compound 10) is dissolved in anhydrous THF (45 mL). Upon addition of triethylamine (168 μL, 1.21 mmol, 1.5 eq.) the solution is cooled to −5° C. A solution of ethyl chloroformate (115.2 μL, 1.21 mmol, 1.5 eq.) in anhydrous THF (45 mL) is added dropwise. The resulting solution is stirred at −5° C. for 1 hour. The solution is allowed to warm to 4° C. upon which a solution of sodium borohydride (153.0 mg, 4.04 mmol, 5.0 eq.) in H₂O (45 mL) is added. After stirring at RT for 16 hours the solution is concentrated in vacuo. The obtained crude product (473 mg) is purified by flash chromatography (solvent system cyclohexane/ethyl acetate 1:1, then cyclohexane/ethyl acetate 2:3, then cyclohexane/ethyl acetate 1:3) to yield the desired benzylic alcohol. The latter is converted to the respective azide. For that, the benzylic alcohol (100 mg, 184.3 μmol) is dissolved in toluene (10 mL). After addition of 1,8-diazabicyclo[5.4.0]undec-7-ene (DBU, 82.7 μL, 553.0 μmol, 3.0 eq.) and diphenylphosphorylazide (DPPA, 51.6 μL, 237.7 μmol, 1.3 eq.) the solution is stirred at RT. After 6 days LC-MS analysis shows complete conversion to the desired azido modified Bodipy.

For the reduction of the azido modified Bodipy to the respective amine, saturated aqueous ammonium chloride solution (10 mL) and zinc powder (602 mg, 9.2 mmol, 50 eq.) are added to the solution above. After stirring at RT for 16 hours additional toluene (10 mL), H₂O (10 mL) and zinc powder (602 mg, 9.2 mmol, 50 eq.) are added. The suspension is stirred for further 16 hours upon which additional zinc powder (602 mg, 9.2 mmol, 50 eq.) is added. After stirring at RT for 2 days, additional zinc powder (1000 mg, 15.3 mmol, 83 eq.) is added and stirring is pursued at 40° C. for 4 days. Saturated sodium bicarbonate solution (20 mL) is added and the zinc removed by filtration through a paper filter. After extraction of the resulting solution with DCM (3×20 mL), the combined organic layers are dried over Na₂SO₄, filtered and evaporated under reduced pressure to yield the crude product (128 mg). A part of the crude product (89.2 mg) is subjected to HPLC purification. This yields the desired amine.

For the conversion of the amine to the respective chloroacetamide, the crude amine (38.3 mg, 55.2 μmol) is dissolved in DMF (300 μL). DIPEA (29.0 μL, 166 μma 3.0 eq.) and chloroacetic anhydride (18.9 mg, 110.5 μmol, 2.0 eq.) are added and the resulting solution is stirred at RT for 150 minutes. Additional DIPEA (29.0 μL, 166 μma 3.0 eq.) and chloroacetic anhydride (9.45 mg, 55.25 μmol, 1.0 eq.) are added and the solution stirred at RT for further 16 hours. Additional DIPEA (14.5 μL, 83 μma 1.5 eq.) is added and the solution is stirred at RT for further 16 hours. Upon acidification with acetic acid (30 μL) the solution is subjected to repeated HPLC purification. This yielded the target chloroacetamidomethyl modified.

[M+H]+ₜₕₑₒ: 618.28 [M+H]+_{found}: 618.4

### Example 6

The procedure of Example 1 for preparing the appropriate peptide is repeated, but the compound of Example 4 is used in the last step instead of Bodipy-CO—NH—(CH₂)₂-Maleimide, in accordance with the following scheme:

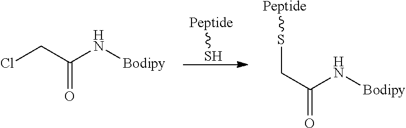

The peptide that is containing a thiol group (1-50 μmol, 1-5 eq.) is suspended in 100 mM aqueous ammonium hydrogen carbonate/ACN/NMP (1:4:1 or other amounts that lead to at least partial dissolution of the peptide). The compound of Example 4 (1-50 μmol, 1-5 eq.) is added and the reaction is stirred for 1-72 hours at a temperature of 20-50° C. It may be required to add further amounts of any of the starting materials or solvents followed by longer incubation times to drive the reaction to completion. When LC-MS analysis shows that sufficient amounts of target product have been formed, the crude product is obtained by filtration, centrifugation and decantation of the overlaying solvent, concentration in vacuo, or extraction with solvents like dichloromethane or ethyl acetate. The crude peptide is dissolved (preferably in DMSO or mixtures of ACN and water) and purified by HPLC, thus yielding the peptide with the desired fluorophore linked to it, as indicated above.

The peptide used as starting material (Peptide —SH) may be one of the following peptides:

H-DAEFRHDSGYEVHHQ-Lys(-CO-CH₂-SH)-LVFFAEDVGSNKGAI
IGLMVGGVVIA-OH
H-DAEFRHDSGYEVHHQ-Lys(-CO-CH₂-SH)-LVFFAEDVGSNKGAI
IGLMVGGVV-OH
H-DAEFRHDSGYEVHHQ-Lys(-CO-CH₂-SH)-LVFFAEDVGSNKGAI
IGLMVGG-OH
H-DAEFRHDSGYEVHHQ-Lys(-CO-CH₂-SH)-OH
H-Lys(-CO-CH₂-SH)-LVFFAEDVGSNKGAIIGLMVGGVVIA-OH
H-QSHYRHISPAQVHHQ-Lys(-CO-CH₂-SH)-OH,
and
H-RPRTRLHTHRNRHHQ-Lys(-CO-CH₂-SH)-OH

### Example 7: Palmitoyl-X-KFFVLK-NH₂

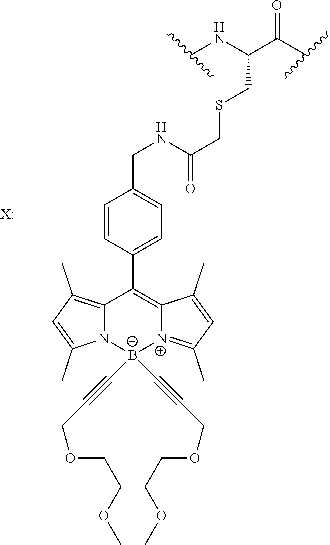

a) Solid phase peptide synthesis: Palmitoyl-Cys-KFFVLK-NH₂

For peptide synthesis the following amino acid derivatives are used: Palmitic acid, Fmoc-Cys(Trt)-OH, Fmoc-Lys(Boc)-OH, Fmoc-Phe-OH, Fmoc-Val-OH, Fmoc-Leu-OH, Fmoc-Gly-OH.

Peptide synthesis is performed using a Syro II peptide synthesizer (MultiSynTech, Witten, Germany) using the following conditions (synthesis scale: 0.3 mmol): Polystyrene AM RAM (Rapp Polymere, Tubingen, Germany) (0.3 mmol) is subjected to Fmocdeprotection with a solution of piperidine in DMF (15:85, 2× 10 min). Repeating cycles of coupling of Fmoc protected amino acids and Fmoc cleavage are performed until the linear target peptides are assembled. For the coupling of the amino acids the following reagents are employed: Fmoc-AA-OH (4 eq., AA=suitably side chain protected amino acid), OxymaPure (1.0 eq.), DIC (4 eq.) in DMF for 60 min. For all amino acids double couplings are performed. All Fmocdeprotection steps are done with a solution of piperidine in DMF (15:85, 2× 10 min). After finalization of the synthesis, the peptide is cleaved off the solid support with TFA/H2O/TIPS/EDT (90:3:4:3, RT, 3 hours) and treated with diethyl ether at 0° C. The suspension is centrifuged and the supernatant removed.

b) Purification of the crude peptide

Purification is performed by preparative HPLC (DionexUltiMate 3000 HPLC system) using a PLRP-S column (100 Å, 8 uM, 150×25 mm) at a flow rate of 40 mL/min with a gradient of 15-85% solvent B in 60 min (solvent A: 0.1% TFA in water, solvent B: 0.1% TFA in MeCN) and UV detection at 220 nm. After lyophilization, the peptide is stored at −20° C.

[M+H]+ₜₕₑₒ: 1121.7 [M+H]+_{found}: 1121.1

c) Attachment of chloroacetamidomethyl modified Bodipy to the peptide

The peptide Palmitoyl-Cys-KFFVLK-NH2 (36.0 mg, 32.10 μmol, 1.2 eq.) is suspended in a mixture of 100 mM aqueous ammonium hydrogen carbonate solution (2 mL), MeCN (8 mL), and NMP (2 mL). Chloroacetamidomethyl modified Bodipy (16.5 mg, 26.70 μmol, 1.0 eq.) is added and the suspension stirred at RT for 16 hours. The stirring is continued for further 4 days at RT and additional 2 days at 40° C. 100 mM aqueous ammonium hydrogen carbonate solution (2 mL) is added and stirring continued at 50° C. The obtained suspension is centrifuged and the solid material subjected to purification by HPLC. The same conditions as for the peptide purification are used with the following exceptions: C18 column (Kromasil 100-5C18, 250×21.2 mm), gradient 50-95% solvent B in 60 min.

Purity: 90.3% [M+H]+ₜₕₑₒ: 1703.1 [M+H]+_{found}: 1703.0

### Example 8:Palmitoyl-KKFFVLKG-X—NH₂

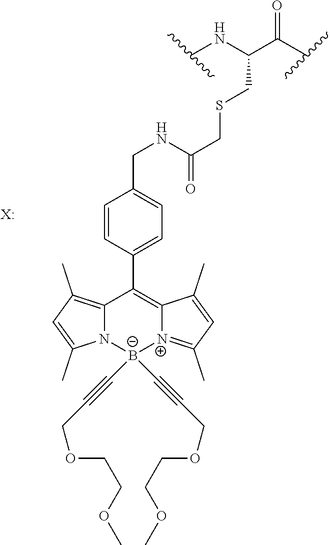

a) Solid phase peptide synthesis: Palmitoyl-KKFFVLKG-Cys-NH₂

For peptide synthesis the following amino acid derivatives were used: Palmitic acid, Fmoc-Cys(Trt)-OH, Fmoc-Lys(Boc)-OH, Fmoc-Phe-OH, Fmoc-Val-OH, Fmoc-Leu-OH, Fmoc-Gly-OH. By following the procedure of Example 7a, Palmitoyl-KKFFVLKG-Cys-NH₂ is obtained.

b) Purification of the crude peptide

Purification is performed as disclosed in Example 7b.

[M+H]+ₜₕₑₒ: 1306.9 [M+H]+_{found}: 1306.1
c) Attachment of chloroacetamidomethyl modified Bodipy to the peptide

The peptide Palmitoyl-KKFFVLKG-Cys-NH2 (36.0 mg, 32.10 μmol, 1.2 eq.) is suspended in a mixture of 100 mM aqueous ammonium hydrogen carbonate solution (2 mL), MeCN (8 mL), and NMP (1 mL). Chloroacetamidemethyl modifiedBodipy (14.2 mg, 22.98 μmol, 1.0 eq.) is added and the suspension stirred at RT for 16 hours. The stirring is continued for further 4 days at RT and additional 2 days at 40° C. 100 mM aqueous ammonium hydrogen carbonate solution (2 mL) is added and the mixture stirred for further 16 hours at 50° C. Further peptide Palmitoyl-KKFFVLKG-Cys-NH2 (7.5 mg, 6.65 μmol, 0.25 eq.) is added and the mixture stirred for further 4 days at 50° C. The obtained suspension is centrifuged and the solid material subjected to purification by HPLC. The same conditions as for the peptide purification are used with the following exceptions: C18 column (Kromasil 100-5C18, 250×21.2 mm), gradient 40-100% solvent B in 60 min.

Purity: 93.7% [M+H]+ₜₕₑₒ: 1888.2 [M+H]+_{found}: 1888.1 Yield: 0.1 mg (0.2%)

### Example 9: Synthesis of Compound of Formula

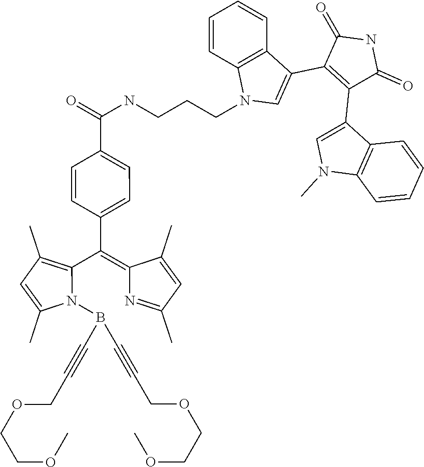 [SMILES: COCCOCC#CB(C#CCOCCOC)n1c(C)cc(C)c1/C(=C1\N=C(C)C=C1C)c1ccc(C(=O)NCCCn2cc(C3=C(c4cn(C)c5ccccc45)C(=O)NC3=O)c3ccccc32)cc1]

A mixture of 2-(N-methyl-indolyl)-3-(N-phthalimidoyl-indolyl)-maleimide and hydrazine monohydrate in EtOH (10 ml) is refluxed overnight. The volatiles are evaporated under vacuum. The residue is dissolved in a mixture of DCM/MeOH (9/1) and then filtered. The resulting solution is washed twice with water. The organic layer is dried over MgSO₄ filtered and evaporated under vacuum. The residue is dissolved in dry DCM (10 ml) (Solution A).

A solution of Bodipy-CO—OH, N-(3-dimethylaminopropyl)-N′-ethylcarbodiimide hydrochloride, 1-hydroxybenzotriazole and N,N-dimethylpyridin-4-amine in dry DCM (15 ml) is stirred 1h at rt. The resulting solution is dropped into Solution A by cannula. The mixture is stirred overnight at rt. Water is added and the reaction mixture is extracted 3× with DCM. Combined organic layers are washed 3× with H₂O, dried over MgSO₄, filtered and evaporated under vacuum. The residue is purified by column chromatography (SiO₂, DCM/AcOEt/toluene, 45/45/10) then recrystallized once in AcOEt/Et₂O and once in AcOEt/pentane to yield the title compound as an orange powder.

The starting material Bodipy-CO—OH is prepared as follows:

In a 100 ml two-necked flask equipped with a reflux condenser are placed 50 ml of benzene, 18 ml of tert.-butanol and 4 ml of H₂O. Then 10-(4-iodophenyl)-5,5-bis(3-(2-methoxyethoxy)prop-1-yn-1-yl)-1,3,7,9-tetramethyl-5H-dipyrrolo[1,2-c:2′,1′-f][1,3,2]diazaborinin-4-ium-5-uide (R₁), triethylamine and bis(triphenylphosphine)palladium(II) dichloride are added. Carbon monoxide is fed upon stirring for 7h at 70° C. The reaction is monitored by TLC (SiO₂, AcOEt/EtOH, 9:1). Then the mixture is cooled down and diluted with DCM and H₂O. About 20 ml of HCl (1M) are added to get pH=3-4. The organic layer is washed with 3× H₂O and dried over MgSO₄. The solvent is removed under vacuum. The residue is purified by column chromatography (AcOEt/PE, 4:6; to take off the first side product, AcOEt/AcOH, 99:1) and recrystallized from DCM/EtOH. The compound is purified by column chromatography (SiO₂, AcOEt/EtOH 9/1) to yield a green orange solid.

In the following Examples, cytometer platforms equipped with lasers and optical filters (e.g. Navios from Beckmann Coulter equipped with 3 lasers, 10 colors) are used to measure separately or simultaneously one or more fluorescence signals using channels selected according to the emission profile of each test compound of the invention: e.g. the channel FL1 for measurement of fluorescence intensity when the test compound′ emission peaks at 520 nm (a compound of Ex.1) and the channel FL3 for measurement of fluorescence intensity when the test compound′ emission peaks at 650 nm (a compound of Ex.9), or both of them in parallel in case of analysis of two samples or simultaneously if both compounds are co-incubated with the same sample to doubly stain the tested cells.

### Example 10 Å:Staining of Animal and Human Erythrocytes (Red Blood Cells: RBCs)

The blood from an animal or a human subject is sampled in specific 5 mL tube, such as lithium-heparin tube. The blood is manually mixed by inversion before use.

Red blood cells (RBCs) are obtained from whole blood after centrifugation at 200 g for 10 minutes at room temperature. RBCs pellet down and segregate from white blood cells, platelets and plasma. 5 μL of RBCs are pipetted, diluted 1000 times in 5 mL of Flow cytometry buffer (FACS buffer: 150 mM of NaCl; 1 mM of D-Glucose; 5 mM of Na₂HPO₄; 0.5 mM CaCl₂) and either numbered by manual counting on disposable Malassez-like counting cell (X100 Cellule Fast Read, Fisher Scientific, Ref #11762712) with a microscope or counted with Flow count Fluorospheres (Beckman, Ref 7547053) so as to obtain a cell concentration of stock solution to be used in the test. This cell concentration will be used to pipette the exact number of cells needed for the assay to reach the precise targeted cell concentration per cytometer tube, e.g. 50 000 cells per mL, 100 000 cells per mL, 200 000 cells per mL, 400 000 cells per mL or 800 000 cells per mLin FACS buffer.

These cells are stained with compounds of the invention at different concentrations for each. The compounds are solubilized in DMSO to obtain different concentration stocks.

From these different concentration stocks, the same quantity (4) of compounds at desired concentration to be tested is added to erythrocytes in each FACS vial to obtain the same % of DMSO (0.5% of DMSO) at the end. The staining lasts 20 min at room temperature in the dark before starting flow cytometry analysis. The staining duration can also last longer times such as 40 min, 1h, 2h, 4h or longer; and cells are washed or not with the FACS buffer before acquisition. In all experiments, a control is performed with the final amount of compound solvent (DMSO alone, without test compound), in order to obtain the auto-fluorescence of the RBCs.

A specific protocol of acquisition of RBCs (e.g. 5 000, 10 000, 20 000, 30 000 cells or 50 000 cells) on the flow cytometer is designed to determine the fluorescence intensities of RBCs stained with each compound: for example for the compounds of Example 1 or 3,the fluorescence is measured in the FL1 channel and for the Compound of Ex.9,the fluorescence is measured in the FL3 channel. These fluorescences are detected by the gating of erythrocytes in a log FSC/log SSC plot.

Each data point is the mean of triplicate or quadruplicate values, meaning that for a tested compound concentration, 3 or 4 repetitions are performed. Results are expressed as Median Fluorescence Intensity (MEDFI). Diverse methods are used to normalize the results of RBC staining. (i) raw data of MEDFI with no calibration; (ii) the RBC auto-fluorescence background is subtracted from the value of MEDFI obtained with each concentration of each corresponding compound (MEDFI-Background); (iii) the MEDFI is divided with the value of the RBC auto-fluorescence background (MEDFI/Background); (iv) Relative mean is also calculated by subtracting the Background value from the MEDFI obtained with the compound and dividing by the mean of the both values ((MDFI−Background)/Mean(MEDFI, Background)).

The percent (%) events in RBC gate is measured with and without incubation with the tested compound and the ratio of the % events in RBC gate in presence of tested compound to the % events in RBC gate in the absence of compound (or the presence of the compound solvent) is used as an index of percent of erythrocyte cell viability.

In studies comparing intensities of the staining of RBCs from Alzheimer patients and healthy controls or subjects with anon-Alzheimer disease or from animal models, predictive modeling with logistic regression model and classification is performed. ROC (Receiver Operating Characteristic) Curve(s) enabling to evaluate the assay accuracy (specificity and sensitivity of the assay). To generate a ROC analysis, a logistic model is used to give a score to each patient. At each threshold between the minimum and the maximum of the population score, a matrix based on the prediction of the model compared to the real data is constructed. Based on these matrices, the accuracy, the sensitivity, the specificity, the negative and the positive predictive value are calculated. With the sensitivity and the specificity, the ROC curve is established and the AUROC (Area Under the Receiver Operating Characteristic) is calculated for the tested compound of the present invention targeting respective biomarker: PKC or Aβ (Aβ1-42 or shorter fragments). The same procedure is applied for the combination of two or more compounds tested. In this case, the class of the patients is indicated with the logical operators (AND, OR). Individual cut-off value from logistic regression model is calculated for each biomarker stained with a compound of the present invention. The score of the model when the accuracy is maximum is taken and with the equation of the model to reversely calculate the concentration of the biomarker which corresponds to the cut-off value.

### Example 10B: Staining of Animal and Human Erythrocytes (Red Blood Cells: RBCs) after Fixation

The experiment is performed as in Example 10A except that RBCs are prepared at different time-points after blood sampling (from day 1 to month 6 in case of cells fixed) after fixation. Fixation is performed using a fixative solution such as 0.01% to 1% Histofix or 0.01% to 4% paraformaldehyde-containing FACS buffer for a duration of 5 min to several days or weeks, or performed by transferring the cells in new special tubes or capillaries containing such a fixative solution. The results are normalized and analyzed as described in Example 10 Å.

### Example 10C: Staining of Animal and Human Erythrocytes (Red Blood Cells: RBCs) with Two Compounds Simultaneously

The blood is collected, and the erythrocytes prepared and stained as described in Example 10A or 10B, except that the staining is performed by co-incubation with 2 compounds simultaneously. The staining intensities are quantified using only one cytometer channel (FL1 or FL3 or another one), or two cytometer channels, according to respective profile of emission/absorbance spectra of the tested compounds. The results are expressed as MEDFI, normalized and analyzed as described in Example 10 Å.

### Example 11 Å: Staining of Animal and Human White Blood Cells (WBCs)

The blood from an animal or a human subject is sampled and mixed as disclosed in Ex. 10 Å. 3 mL of whole blood are added in a 50 mL falcon containing 22 mL of PBS 1×. After centrifugation (e.g. 7 min at 1400 g), the supernatant is removed. The blood is washed a second time with 10 mL of PBS 1×. The cell pellet is re-suspended with 40 mL of lysis buffer (BD Pharm lyse, 10×). After 15 min of incubation in the dark, the solution is centrifuged 5 min at 1400 rpm. The cell pellet is washed 2 times with 15 mL of FACS buffer (150 mM of NaCl; 1 mM of D-Glucose; 5 mM of Na₂HPO₄; 0.5 mM CaCl₂). The pellet is re-suspended with 1 mL of FACS buffer containing serum albumin (such as BSA) at 0%, 0.1%-1%. The cells are counted and a cell solution is prepared with 100 000 cells per mL in FACS buffer containing or not serum albumin.

The WBC cells are stained with a compound of the present invention at different concentrations.

The compounds are solubilized in DMSO to obtain different concentration stocks. From these different concentration stocks, the same quantity (μL) of the compound is added to the WBC preparation in each FACS vial to obtain the same final % of DMSO of 0.5% for all tested tubes. The staining of WBCs with the compound lasts 20 min at room temperature in the dark. The staining duration can also last longer times such as 40 min, 1 h, 2h, 4h or longer; and cells are washed or not with the FACS buffer before acquisition. The experiment is performed the same day of blood sampling (and/or at tested times of day 0 to month 6 in case of fixed cells). Fixation of cells is performed using a fixative solution such as 0.01% to 1% Histofix or 0.01% to 4% paraformaldehyde-containing FACS buffer for a duration of 5 min to several days or up to month 6, or performed by transferring the cells in new special tubes or capillaries containing such a fixative solution.

In all experiments, a control is performed with the final amount of compound'solvent (DMSO alone, without compound) to obtain the auto-fluorescence of the WBCs.

The protocol of acquisition is designed to determine the fluorescence of the WBCs, including lymphocytes, granulocytes, monocytes, stained with the 2 compounds. To gate the WBC population, a control tube containing the desired concentration of WBCs stained with the CD45-Krome orange (Beckman coulter as specified in Ex. 10 Å) in final volume of 0.5 mL is used.

The compound of Ex. 1 and the compound of Ex. 3 produce fluorescence in FL1 channel and the Compound of Ex.9 produces the fluorescence in FL3 channel. The CD45 Krome-orange produces fluorescence in FL10 channel. These fluorescences are detected by the gating of WBCs in a log FSC/log SSC plot. Different cell populations, including lymphocytes, granulocytes and monocytes are gated.

Each data point is the mean of triplicate or quadruplicate values, meaning that for a tested compound concentration, 3 or 4 repetitions are performed. The results are expressed as MEDFI, normalized and analyzed as described for RBCs in Example 10 Å.

### Example 11B: Staining of Animal and Human White Blood Cells (WBCs) after Fixation

The experiment is performed as in Example 9A except that WBCs are prepared at different time-points after blood sampling (from day 1 to month 6 in case of cells fixed) after fixation. Fixation is performed using a fixative solution such as 0.01% to 1% Histofix or 0.01% to 4% paraformaldehyde-containing FACS buffer for a duration of 5 min to several days or weeks, or performed by transferring the cells in new special tubes or capillaries containing such a fixative solution. The results are expressed as MEDFI, normalized and analyzed as described in Example 10 Å.

### Example 11C: Staining of Animal and Human White Blood Cells (WBCs) with Two Compounds Simultaneously

The blood is collected, and the white blood cells (WBCs) prepared and stained as described in Ex. 9A or 9B, except that the staining is performed by co-incubation with 2 compounds simultaneously. The staining intensities are quantified using only one cytometer channel (FL1 or FL3 or another one) or two cytometer channels, according to respective profile of emission-absorbance spectra of the tested compounds. The results are expressed as MEDFI, normalized and analyzed as described in Example 10 Å.

### Example 12: Dose-Dependent Staining of RBCs with the Compounds of the Present Invention

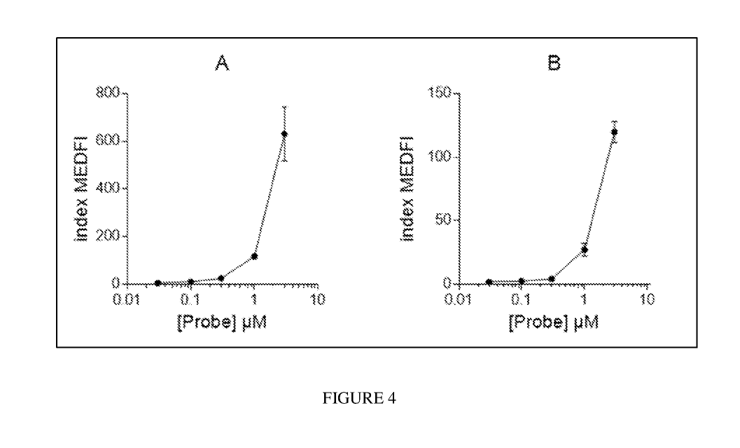

Using the method of Example 10 Å, compound of Ex. 1 and compound of Ex.9 are tested each at 0.03, 0.1, 0.3, 1 and 3 μM concentrations to establish corresponding dose-dependent staining of red blood cells (RBCs: 200 000 cells/mL) from human subjects. Each concentration of Compound of Ex. 1 and each concentration of Compound of Ex.9 is tested separately in quintuplicate and the RBC staining is measured using the cytometer channel FL1 and FL3, respectively. FIG. 4 shows the results obtained on RBCs of 3 human subjects. These results show a dose-dependent staining of red blood cells by compound of Ex. 1(FIG. 4A) and by compound of Ex.9 (FIG. 4B).

### Example 13: Upper Limit of Use Value for Compounds of the Present Invention

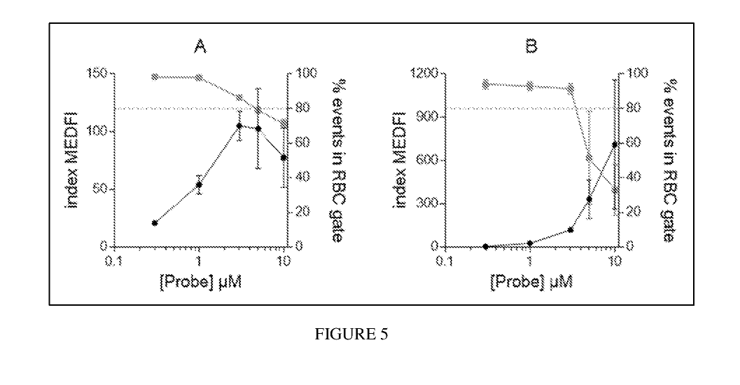

Using the method of Example 10 Å, in order to determine these Upper Limit of Use values in the cellular assay of RBCs (RBCs:200 000 cells/mL) from the intended use population (patients with Alzheimer's disease or aged-matched controls) are used to select an optimal dose range of each compound. Triplicates for each donor are prepared and used. The compounds are tested not only at concentrations of below 3 μM (as in FIG. 4) but also at higher concentrations i.e. 5 μM and 10 μM, for their ability to stain the RBCs and also for their potential to impact the survival of RBCs by measuring the % events in RBC gate. The Upper Limit of Use value is determined in this specific RBC-based test as the compound concentration that leads to the highest intensity of staining (MEDFI) without a significant effect on or an effect of less than 20% decrease on the percent events in RBC gate. Results from experiments on RBCs from 3 subjects (FIG. 5) indicate that compound of Ex. 1 (FIG. 5A) and compound of Ex. 9 (FIG. 5B) can be used at concentrations ranging up to the high concentration of 3 μM in the RBC assay. The Upper Limit of Use value for each of these compounds is 3 μM.

### Example 14: Determination of Lower Limit of Detection (LOD) and Lower Limit of Quantification (LLOQ) of the Assay Using with the Compounds of the Present Invention

Using the method of Example 10 Å, RBCs (200 000 cells/mL) from 5 human subjects are processed without any compound to determine the limit of detection (LOD). In parallel, a concentration range of each compound is used to determine the Lower Limit of Quantification (LLOQ) i.e. the lowest level of measure and that can be detected with an acceptable level of imprecision. All experiments are done in triplicates. The mean and standard deviation of the 125 measures are used to calculate the LOD according to the following equation: LOD=Mean+(3.29×SD). For LLOQ determination, variation coefficients are calculated for each concentration of compound and expressed in percentage according to the following formula:

$$\% CV = \frac{σ}{μ} \times 100 ,$$

where σ is me standard deviation of the 5 replicates values and μ the mean of the 5 replicates values.

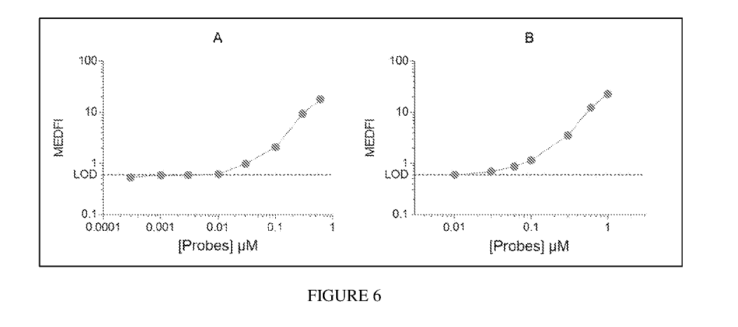

The results show that the LOD is 0.604 for compound of Ex. 1 (FIG. 6A) and 0.452 for compound of Ex. 9 (FIG. 6B). The LLOQ is 0.616 and 0.598 for compound of Ex. land compound of Ex. 9, respectively (Table 1). The LLOQ values are obtained with the concentration 0.01 μM for both compounds of Ex. land Ex. 9.

TABLE 1
Values of LOD and LLOQ for Compounds of Ex. 1 and 9.

| | Compound Ex. 1 | Compound Ex. 9 |
|---|---|---|
| LOD | 0.604 | 0.452 |
| LLOQ | 0.616 | 0.598 |
| Compound concentration | 0.01 μM | 0.01 μM |
| to obtain LLOQ | | |

### Example 15: Effect of Concentrations of RBCs on the Intensity of their Staining by the Compounds of the Present Invention

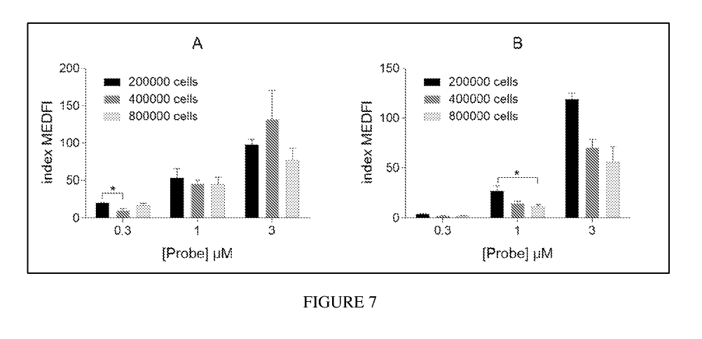

Using the method of Example 10 Å, the Compound of Ex. 1 (FIG. 7A) and Compound of Ex. 9 (FIG. 7B) are tested at 0.3, 1 or 3 μM to stain different concentrations of red blood cells: 100 000, 200 000, 400 000 or 800 000 cells/mL from human subjects and in triplicates. The results show that the staining is optimal for 100 000 cells and 200 000 cells/mL for both Compound of Ex. land Ex. 9. However, the staining decreases with increased number of cells (to 400 000 cells/mL or 800 000 cells/mL). Therefore, under the experimental conditions used here, the concentrations of 200 000 cells/mL and below leads to optimal staining of RBCs with Compound of Ex. 1 or Ex. 9 tested.

### Example 16: Studies in Alzheimer Animal Models

Using the method of Example of 10A to measure the staining of RBCs with the compound of Ex.1 specific to Aβ biomarker and the compound of Ex.9 specific to PKC biomarker, this cellular assay of RBCs is then used in animal models of Alzheimer's disease versus control animal groups and in clinical studies recruiting patients with early mild or moderate to severe Alzheimer's disease versus age- and gender-matched control human subjects (either healthy subjects or subjects with a non-Alzheimer neurodegenerative disease).

The results show that such an RBC assay, using for example the compound of Ex.1, the compound of Ex.9 and their combination, enables to discriminate with a good accuracy between the transgenic mouse group and control mouse group.

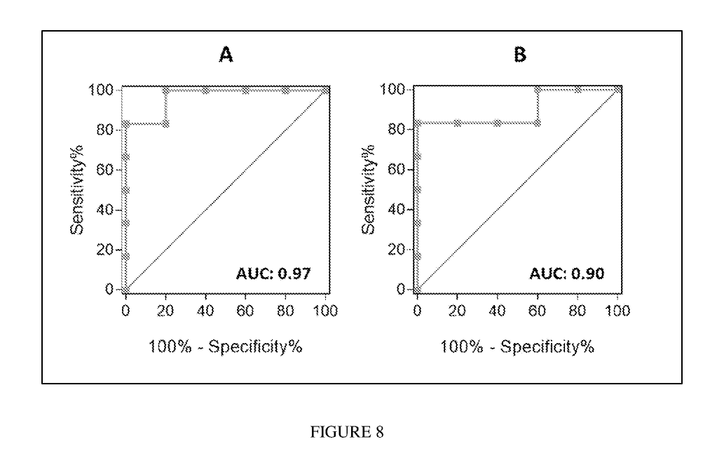

FIG. 8 on the results from a study on RBCs from 11 adult mice (5 transgenic mice and 6 age-matched wild type mice) shows that the use of the compound of Ex. 1 (1 μM) or the compound of Ex.9 (1 μM) enables to discriminate between the two groups with an excellent accuracy. Transgenic mice used in this study carry two mutations associated with early onset AD: mutant amyloid precursor protein (K670N and M671L) and mutant human presenilin 1 (M146V). These transgenic mice develop AD-related phenotype consisting in beta-amyloid peptide overproduction and formation of amyloid plaques in their brain.

### Example 17: Clinical Studies Establishing the Correlation Between the Staining with Compounds of the Present Invention of RBCs of Alzheimer and Control Subjects

### Congruence Coefficient

TABLE 2
Congruence coefficient

| | | | Ratio: | | |
|---|---|---|---|---|---|
| | | Raw data | MEDFI/ | MEDFI- | Relative |
| Compound | Compound | MEDFI | Background | background | mean |
| Ex. 1 | Ex. 3a | 0.812 | 0.786 | 0.810 | 0.816 |
| Ex. 1 | Ex. 3b | 0.819 | 0.727 | 0.818 | 0.814 |
| Ex. 3a | Ex. 3b | 0.990 | 0.921 | 0.990 | 0.998 |

Using the method of Example 10 Å, the compounds are screened at 0.3, 0.6 and 1 μM in triplicates to stain RBCs from patients with Alzheimer's disease or age-matched healthy controls (8 to 30 subjects depending on experiments), and the different normalization methods of the data before the calculation of the correlation factors for each two compounds and for each concentration. In addition, the congruence coefficient as an index of the similarity between the profile of two compounds is also calculated taking into account the staining obtained at the 3 tested compound concentrations.

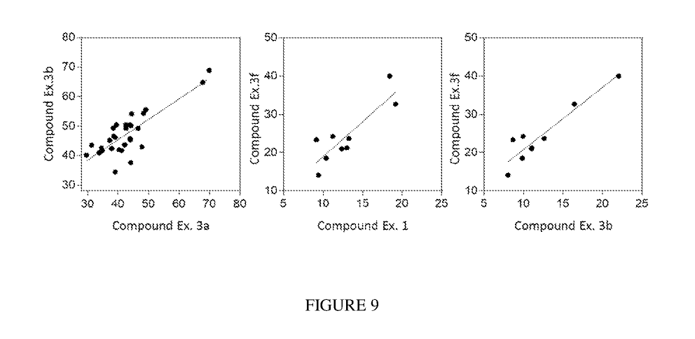

The results show that the RBC staining with the compounds of Ex. 1, Ex. 3a, Ex. 3b well correlate as exemplified in FIG. 9. Their correlation factor reaches above 0.8 (FIG. 9) and their congruence factor reaches up to 0.998 (Table 2), depending on the result normalization method practiced. The RBC staining with compounds of Ex. 1, 3a and 3b also correlate to the RBC staining with compound of Ex. 3f (FIG. 9).

### Example 18: Correlation Between the RBC Staining with Compounds of the Present

invention and the level of the cerebrospinal biomarkers Aβ42, tau and phosphorylated-tau in patients with Alzheimer's disease

Using the method of Example 10 Å, the staining level (obtained with the compounds of the present invention, each tested at 0.01 to 3 μM in triplicates) of RBCs from patients with Alzheimer's disease is compared to the cerebrospinal fluid (CSF) level of the Aβ42 measured in the same patients (CSF Aβ42 is measured using immunodetection assay, as a biomarker for diagnosis of AD patients who had a lumbar puncture to collect the CSF, in the hospital).

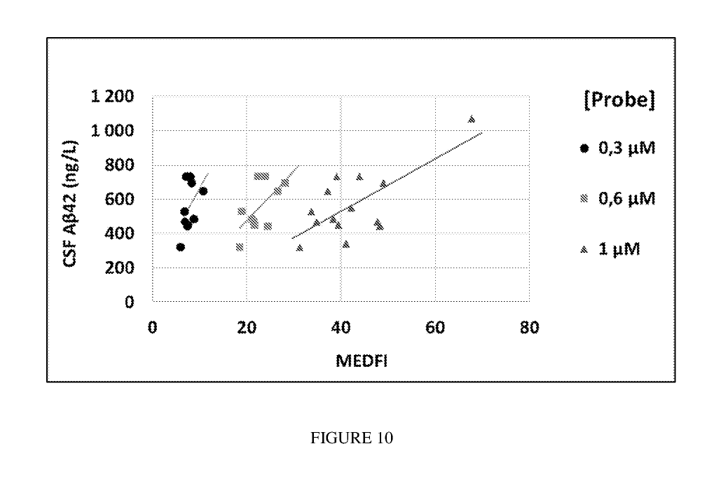

The results show a good correlation between the fluorescence intensity of RBCs stained with the compound of Ex. 3a and the level of cerebrospinal fluid Aβ42 (FIG. 10).

Thus, the results obtained with the compounds and the non-invasive method of the present invention correlate with the results of CSF Aβ42 that implicates the invasive procedure of lumbar puncture for sample collection.

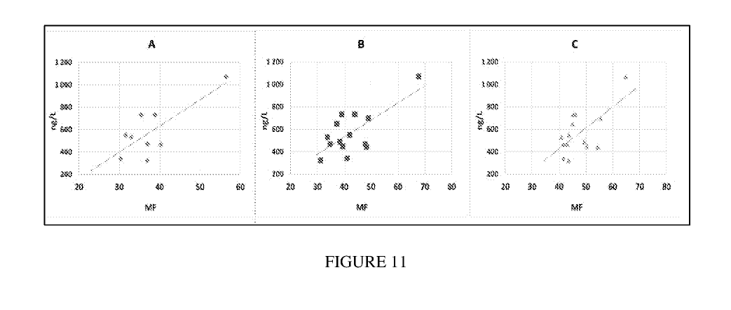

The staining level obtained with the other compounds of the present invention of RBCs from patients with Alzheimer's disease is also compared to the cerebrospinal fluid level of Aβ42 biomarker and the results show a direct correlation (FIG. 11) or an inverse correlation depending on which compound tested.

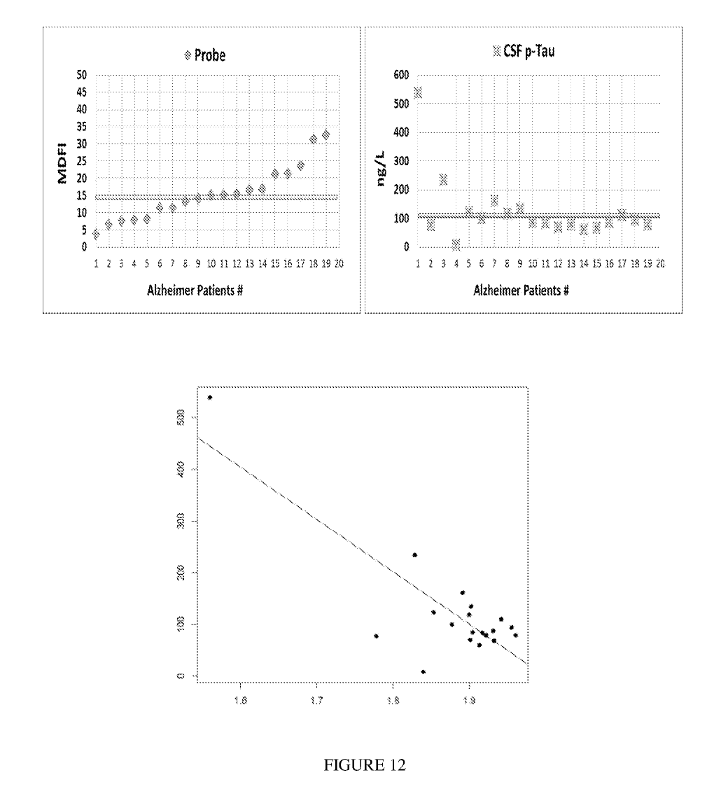

The staining level obtained with the compounds of the present invention (tested at concentrations of 0.01 to 3 μM) of RBCs from patients with Alzheimer's disease is also compared to the cerebrospinal fluid level of the tau and phosphorylated tau biomarkers and the results show correlation or inverse correlation depending on which compound of the invention tested. FIG. 12 shows the result of an inverse correlation between the CSF level of phosphorylated-tau and the level of staining of erythrocytes of the same Alzheimer patients with the compound of Ex. 9 used at 104.

SEQUENCE LISTING

| | |
|---|---|
| DAEFRHDSGYEVEIHQK | SEQ ID NO: 1 |
| LVFFAEDVGSNKGAIIG | (Human Aβ1-42 peptide) |
| LMVGGVVIA | |
| QSHYRHISPAQVEIHQK | SEQ ID NO: 2 |
| | (D-enantiomeric peptide D1) |
| RPRTRLHTHRNRHHQK | SEQ ID NO: 3 |
| | D-enantiomeric peptide D3 |
| CKFFVLK-NH₂ | SEQ ID NO: 4 |
| | (Based on the core sequence of |
| | Aβ16-20: KLVFF) |
| KKFFVLK-NH₂ | SEQ ID NO: 5 |
| | (Based on the core sequence of |
| | Aβ16-20: KLVFF) |
| KKFFVLKGK-NH₂ | SEQ ID NO: 6 |
| | (Based on the core sequence of |
| | Aβ16-20: KLVFF) |
| KKFFVLKGC-NH₂ | SEQ ID NO: 7 |
| | (Based on the core sequence of |
| | Aβ16-20: KLVFF) |
| KKFFVLKG-NH₂ | SEQ ID NO: 8 |
| | (Based on the core sequence of |
| | Aβ16-20: KLVFF) |

1. A compound of formula I:
P—Ar—R—X-T (I)
wherein
P is a residue of formula (a) 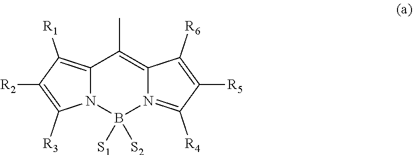
wherein
each of R₁, R₂, R₃, R₄, R₅ and R₆ is, independently, H or C₁-C₁₀alkyl, and
each of S₁ and S₂ is, independently, a hydrophilic group of formula —C≡C-L-A wherein L is a single bond, C₂-C₄alkenylene or linear, branched saturated C₂-C₂₀ carbon chain interrupted by 1 to 10 oxygen atoms; and A is C₁-C₄ alkyl, a phosphate group or a sulfonate group;
Ar is C₅-C₁₄arylene or heteroarylene on which —R—X-T is in ortho, meta or para position;
R is —CO—NH—, —NH—CO—, —CH₂—CO—NH— or —CH₂—NH—CO—; and
either X is a spacer serving to distance the residue T from the residue P without affecting the fluorescence of P and the biological activity of T, X being covalently bound through —S— to the α side chain of an amino-acid forming part of the residue T; and T is a peptidic residue selected from a residue of
an Aβ peptide,
QSHYRHISPAQVHHQK,
RPRTRLHTHRNRHHQK,
CKFFVLK-NH₂
KKFFVLK-NH₂,
KKFFVLKGK-NH₂,
and
KKFFVLKGC-NH₂
or a derivative or a structural analog thereof, comprising at least one amino acid with an α side chain;
or X is a spacer serving to distance the residue T from the residue P without affecting the fluorescence of P and the biological activity of T; and T is a group of formula (b)

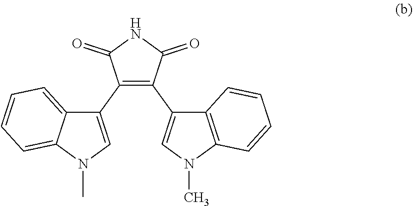 [SMILES: Cn1cc(C2=C(c3cn(C)c4ccccc34)C(=O)NC2=O)c2ccccc21]

2. A compound according to claim 1, of formula Ia
P—Ar—R—Xₐ-Tₐ (Ia)
wherein P, Ar and R are as defined in claim 1,
Xₐ is a spacer serving to distance the residue Tₐ from the residue P without affecting the fluorescence of P and the biological activity of Tₐ, Xₐ being covalently bound through —S— to the α side chain of an amino-acid forming part of the residue Tₐ; and
Tₐ is a peptidic residue T as defined in claim 1.

3. A compound according to claim 1, of formula Ib
P—Ar—R_{b}—X_{b}-T_{b} (Ib)
wherein P and Ar are as defined in claim 1, R_{b} is —CO—NH— or —CH₂—CO—NH—; X_{b} is C₂-C₅ alkylene and T_{b} is a group of formula (b) as indicated in claim 1.

4. A complex comprising a compound of Formula I according to claim 1 and a cell or cell membrane, said complex being obtainable by incubation of said cell or cell membrane with 0.0001 to 3 μmolar of a compound of formula I, in a biological fluid or an iso-osmotic medium.

5. A composition comprising one or more reagents selected from the group consisting of:
(a) one or more reagents for isolating or purifying a cell or a cell membrane to which a compound of formula I according to claim 1 has been or will be bound,
(b) one or more reagents for incubating the cells or cell membranes with a compound of formula I according to claim 1,
(c) one or more reagents for enhancing and/or inhibiting the binding and/or interaction of compound of formula I according to claim 1 with a cell or cell membrane, and
(d) one or more reagents for measuring a complex of a compound of formula I according to claim 1 with the cell or cell membrane.

6. A kit comprising a composition according to claim 5, associated with a device to detect or quantify the amount of complex formation, and/or a software for detecting, quantifying or otherwise analyzing complex formation, and/or written instructions or user manual for using the kit to detect or assess the risk of AD.

7. A method for detecting the presence of a complex between a compound of formula I according to claim 1 and cell or a cell membrane comprising the steps of
(a) purifying or isolating cells or cell membranes from a biological sample (e.g., blood sample) of a subject suspected of having, or at risk of developing AD or a disease or disorder characterized by deposits of β amyloid peptides in brain or nervous system;
(b) contacting the purified or isolated cells or cell membranes with a compound of formula I according to claim 1 alone or in the presence of a reagent for enhancing and/or inhibiting the binding and/or interaction of a compound of formula I with a cell or cell membrane, and
(c) detecting complex formation between the cells or cell membranes and the compound of formula I according to claim 1.

8. A method for diagnosing a subject as having AD or for being at risk of developing or progressing for AD or a disease or disorder characterized by the abnormal deposition of β-amyloid and/or tau and/or abnormal PKC changes and/or other proteins downstream to PKC and/or β-amyloid changes comprising:
a) purifying or isolating cells or cell membranes from a biological sample (e.g., blood sample) of a subject suspected of having, or at risk of developing AD or a disease characterized by deposits of β-amyloid peptides in brain or nervous system;
b) contacting the purified or isolated cells or cell membranes with a compound of Formula I of claim 1, e.g. a compound of formula Ia or Ib (e.g., a compound of Ex. 1, Ex. 3a, 3b, or Ex. 9);
c) enhancing and/or inhibiting the binding and/or interaction of a compound of formula I, e.g. a compound of formula Ia or Ib, with a cell or cell membrane, e.g. by addition of an appropriate agent;
d) detecting complex formation between the cells or cell membranes and the compound of formula I, preferably by measuring the fluorescence of compound of formula I, e.g. a compound of formula Ia or Ib, bound to said cells or cell membranes;
e) comparing the amount of complex formation to a reference standard (e.g., normal subject, in a subject not having AD, or to a normal control value), and;
f) diagnosing whether the subject has AD or is being at risk of developing AD when complex formation is higher or lower according to the test compound bound to respective biomarker compared to the reference standard.

9. A method for diagnosing a subject as having AD or for being at risk of developing or progressing for AD or a disease or disorder characterized by abnormal deposition of β-amyloid and/or tau and/or abnormal PKC changes and/or other proteins downstream to PKC and/or β-amyloid changes, comprising:
(a) purifying or isolating cells or cell membranes from a biological sample of a subject suspected of having, or at risk of developing or progressing to, AD or such a disease or disorder,
(b) contacting the purified or isolated cells or cell membranes with a compound of formula Ib according to claim 3, under conditions including an iso-osmotic medium and temperature between 4 and 42° C. and an exposure time, which allows said compound to be bound to cell membranes and/or loaded inside the cells,
(c) contacting the purified or isolated cells with a compound of formula Ia according to claim 2 at a concentration between 0.0001 and 3 μM for a time and under conditions sufficient for the compound of formula Ia to enable sufficient detectable binding signal, and detecting the fluorescent staining of the cells contacted with the compound of formula Ia and formula Ib
(d) comparing the fluorescent staining level of the cells from a patient with AD or another degenerative disease or disorder to the fluorescent staining level of the cells from a normal subject, a subject not having AD, or to a normal control value, or to the fluorescent staining of a positive or a negative calibrator in a stabilized blood sample, and
(e) diagnosing the subject as having AD or other degenerative disease or disorder or as being at risk of developing or progressing for AD or other degenerative disease or disorder when the fluorescent staining level of cells determined for a patient with AD or other degenerative disease or disorder is higher than the fluorescent staining level of cells determined for the normal subject, a subject not having AD, or other neurodegenerative disease or disorder, or normal control value.

10. A method according to claim 8 wherein steps b) and c) are performed using either the same cell population type from the same subject, or different cell population types from the same subject.

11. A method for screening an agent or drug that changes the cell staining by a compound of formula I according to claim 1, for use as a therapeutic agent for AD or for a disease or disorder characterized by β-amyloid and/or tau deposits and/or abnormal changes in PKC, comprising
a) purifying or isolating cells or cell membranes,
b) contacting the isolated cells or cell membranes (i) with an agent or drug to be tested alone, (ii) with the excipient or vehicle of this agent or drug alone, (iii) with 0.0001 to 3 μM of a compound of formula I according to claim 1 alone, and (iv) with the agent or drug to be tested, in the presence of 0.0001 to 3 μM of a compound of formula I according to claim 1 in an iso-osmotic medium, at a temperature of from 4 to 42° C. and an incubation time of from 5 minutes to 24 hours,
c) detecting background or baseline values (only vehicle or excipient alone, only agent or drug alone) and detecting complex formation between the cells or cell membranes and the compound of formula I according to claim 1 by measuring said compound of formula I bound to said cells or cell membranes in the two conditions that are in the absence or the presence of the test agent or drug,
d) subtracting baseline or background values and comparing the resulting amount of complex formation in the cells contacted with the agent or drug and with said compound of formula I to that obtained with said compound of formula I alone,
e) selecting an agent or a drug that changes the complex formation.

12. A method for detecting alterations in a cell or a cell membrane of a circulating or peripheral cell induced by AD or a disease or disorder characterized by deposition of β-amyloid and/or tau and/or by PKC-related abnormalities comprising
a) purifying or isolating cells or cell membranes from a biological sample of a subject suspected of having, or at risk of developing, AD or a disease characterized by deposits of β-amyloid peptide, and/or tau hyper phosphorylation and/or associated with proteinopathy characterized by PKC-related abnormalities,
b) contacting the purified or isolated cells or cell membranes with a compound of formula I according to claim 1 at a concentration between 0.01 and 3 μmolar for a time and under conditions suitable for complex formation, in the presence or the absence of reagent(s) for enhancing and/or inhibiting the binding and/or interaction of a compound of formula I with a cell or cell membrane,
c) detecting complex formation between the cells or cell membranes and the compound of formula I by measuring the fluorescence bound to said cells or cell membranes, and
d) measuring in said isolated cells or cell membranes obtained in step b) a change in the Aβ or its downstream molecular cascade, or changes in PKC conformation or activity or expression or at least one other parameter associated with AD or a disease or disorder characterized by deposition of β-amyloid, and/or tau hyper phosphorylation and/or associated with proteinopathy characterized by PKC-related abnormalities.

13. A method for diagnosing a subject as having a disease or disorder associated with or characterized by the deposit of β-amyloid and/or tau or by abnormal changes in PKC, such as AD, comprising
(a) non-invasively isolating a circulating cell or a peripheral cell of said subject,
(b) detecting an alteration in the membrane of said cell compared to a normal cell, and
(c) diagnosing the subject as having said disease or disorder when the membrane of the cell is altered compared to the membrane of a normal subject not having said disease or disorder.

14. The method of claim 7, wherein the biological sample is a blood sample (e.g., erythrocytes).

15. The method of claim 7, wherein flow cytometry is used to determine the staining/fluorescence of the complex formation between the cells or cell membranes and the compound of formula I (e.g., formula Ia and/or formula Ib).

16. The compound of claim 1, wherein the compound is formula Ia as follows:
H-DAEFRHDSGYEVHHQ-X-LVFFAEDVGSNKGAIIGLMVGGVV-OH
wherein X is:

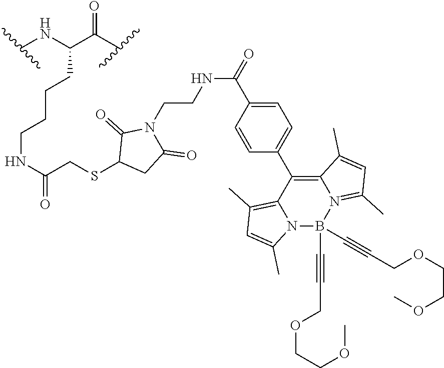

17. The Compound of claim 1, wherein the compound is formula Ia as follows:
H-DAEFRHDSGYEVHHQ-X-LVFFAEDVGSNKGAIIGLMVGG-OH
wherein X is:

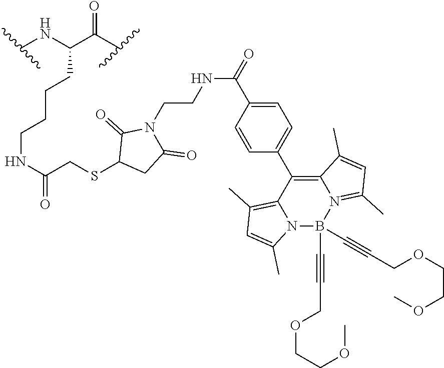

18. The Compound of claim 1, wherein the compound is formula Ib as follows: 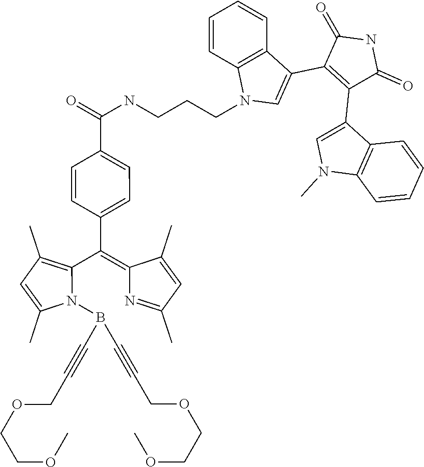 [SMILES: COCCOCC#CB(C#CCOCCOC)n1c(C)cc(C)c1/C(=C1\N=C(C)C=C1C)c1ccc(C(=O)NCCCn2cc(C3=C(c4cn(C)c5ccccc45)C(=O)NC3=O)c3ccccc32)cc1]
